SEQUENCE LISTING (100 sequences)

SEQ ID NO: 1 - DNA, 18 bp, artificial sequence
  aptamer
gggagatagccacgacct

SEQ ID NO: 2 - DNA, 18 bp, artificial sequence
  aptamer
tccaggctgtgcgaaagc

SEQ ID NO: 3 - DNA, 44 bp, artificial sequence
  aptamer
ccgcacaccacgcgcattagcccgcgcacacgacttgaagtagc

SEQ ID NO: 4 - DNA, 44 bp, artificial sequence; identical to SEQ ID NO: 3
  aptamer

SEQ ID NO: 5 - DNA, 43 bp, artificial sequence
  aptamer
cgcacaccacgcgcattagcccgcgcacacgacttgaagtagc

SEQ ID NO: 6 - DNA, 44 bp, artificial sequence
  aptamer
ccgcacaccacgcgcatgaacccgcgcacacgacttgaagtagc

SEQ ID NO: 7 - DNA, 44 bp, artificial sequence; identical to SEQ ID NO: 6
  aptamer

SEQ ID NO: 8 - DNA, 44 bp, artificial sequence; identical to SEQ ID NO: 6
  aptamer

SEQ ID NO: 9 - DNA, 44 bp, artificial sequence; identical to SEQ ID NO: 6
  aptamer

SEQ ID NO: 10 - DNA, 44 bp, artificial sequence
  aptamer
acgcacaccacgcgcatgaacccgcgcacacgacttgaagtagc

SEQ ID NO: 11 - DNA, 44 bp, artificial sequence
  aptamer
ccgcacaccacgcgcatgagcccgcgcacacgacttgaagtagc

SEQ ID NO: 12 - DNA, 44 bp, artificial sequence; identical to SEQ ID NO: 11
  aptamer

SEQ ID NO: 13 - DNA, 44 bp, artificial sequence; identical to SEQ ID NO: 11
  aptamer

SEQ ID NO: 14 - DNA, 45 bp, artificial sequence
  aptamer
gccgcacaccacgcgcatgagcccgcgcacacgacttgaagtagc

SEQ ID NO: 15 - DNA, 43 bp, artificial sequence
  aptamer
cgcacaccacgcgcatgagcccgcgcacacgacttgaagtagc

SEQ ID NO: 16 - DNA, 44 bp, artificial sequence
  aptamer
tcgcacaccacgcgcatgagcccgcgcacacgacttgaagtagc

SEQ ID NO: 17 - DNA, 44 bp, artificial sequence
  aptamer
acgcacaccacgcgcatgagcccgcgcacacgacttgaagtagc

SEQ ID NO: 18 - DNA, 44 bp, artificial sequence; identical to SEQ ID NO: 17
  aptamer

SEQ ID NO: 19 - DNA, 43 bp, artificial sequence
  aptamer
cgcacaccacgcgcatgancccgcgcacacgacttgaagtagc

SEQ ID NO: 20 - DNA, 44 bp, artificial sequence
  aptamer
ccgcacaccacgcgcataatcccgcgcacacgacttgaagtagc

SEQ ID NO: 21 - DNA, 44 bp, artificial sequence
  aptamer
ccgcacaccacgcgcatgaccccgcgcacacgacttgaagtagc

SEQ ID NO: 22 - DNA, 44 bp, artificial sequence; identical to SEQ ID NO: 21
  aptamer

SEQ ID NO: 23 - DNA, 43 bp, artificial sequence
  aptamer
cgcacaccacgcgcgtgaccccgcgcacacgacttgaagtagc

SEQ ID NO: 24 - DNA, 44 bp, artificial sequence
  aptamer
ccgcacaccacgcgcattaacccgcgcacacggcttggagtagc

SEQ ID NO: 25 - DNA, 44 bp, artificial sequence
  aptamer
tcgcacactacgcgcatgaacccgcgcacacgacttgaagtagc

SEQ ID NO: 26 - DNA, 44 bp, artificial sequence; identical to SEQ ID NO: 25
  aptamer

SEQ ID NO: 27 - DNA, 43 bp, artificial sequence
  aptamer
cgcacactacgcgcatgaacccgcgcacacgacttgaagtagc

SEQ ID NO: 28 - DNA, 45 bp, artificial sequence
  aptamer
gccgcacactacgcgcatgagcccgcgcacacgacttgaagtagc

SEQ ID NO: 29 - DNA, 44 bp, artificial sequence
  aptamer
tcgcacactacgcgcatgagcccgcgcacacgacttgaagtagc

SEQ ID NO: 30 - DNA, 44 bp, artificial sequence
  aptamer
ccgcacactacgcgcatgatcccgcgcacacgacttgaagtagc

SEQ ID NO: 31 - DNA, 44 bp, artificial sequence; identical to SEQ ID NO: 30
  aptamer

SEQ ID NO: 32 - DNA, 44 bp, artificial sequence
  aptamer
ctgcacactacgcgcatgatcccgcgcacacgacttgaagtagc

SEQ ID NO: 33 - DNA, 44 bp, artificial sequence
  aptamer
ccgcacgctacgcgcatgaacccgcgcacacgacttgaagtagc

SEQ ID NO: 34 - DNA, 44 bp, artificial sequence
  aptamer
ccgcacactacgcgcacgaacccgcgcacacgacttgaagtagc

SEQ ID NO: 35 - DNA, 44 bp, artificial sequence
  aptamer
tcgcacactacgcgcatgaccccgcgcacacgacttgaagtagc

SEQ ID NO: 36 - DNA, 43 bp, artificial sequence
  aptamer
agcacaccacgcgcataaacccgcgcacacgacttgaagtagc

SEQ ID NO: 37 - DNA, 44 bp, artificial sequence
  aptamer
gagcacaccacgcgcatgaacccgcgcacacgacttgaagtagc

SEQ ID NO: 38 - DNA, 46 bp, artificial sequence
  aptamer
gctagcacaccacgcgcatgaacccgcgcacacgacttgaagtacc

SEQ ID NO: 39 - DNA, 44 bp, artificial sequence
  aptamer
acgcacaccacgcgcatgaacccgcgcacatgacttgaagtagc

SEQ ID NO: 40 - DNA, 44 bp, artificial sequence
  aptamer
ccgcacaccacgcgcattagcccgcgcacacgacttgaagttaa

SEQ ID NO: 41 - DNA, 44 bp, artificial sequence
  aptamer
ccggagacgcgcagctcctatacatgcgcacatgacttgaagtc

SEQ ID NO: 42 - DNA, 44 bp, artificial sequence; identical to SEQ ID NO: 41
  aptamer

SEQ ID NO: 43 - DNA, 44 bp, artificial sequence
  aptamer
cttgagacgcgcatttgctatacatgcgcacgtgacttgaagtc

SEQ ID NO: 44 - DNA, 44 bp, artificial sequence; identical to SEQ ID NO: 43
  aptamer

SEQ ID NO: 45 - DNA, 43 bp, artificial sequence
  aptamer
ctgagacgcgcanttgctatacatgcgcacatgacttgaagtc

SEQ ID NO: 46 - DNA, 46 bp, artificial sequence
  aptamer
ctggagacgcgcagttgctgtacatgcgcacatgacttgaagtagc

SEQ ID NO: 47 - DNA, 44 bp, artificial sequence
  aptamer
gtggagacgcgcatttgctgtacatgcgcacatgactagaagtc

SEQ ID NO: 48 - DNA, 44 bp, artificial sequence
  aptamer
caggagacgcgcatttgttgtacatgcgcacatgactagaagtc

SEQ ID NO: 49 - DNA, 44 bp, artificial sequence
  aptamer
ctggagacgcgcagttgttgtacacgcgcacatgactagaagtc

SEQ ID NO: 50 - DNA, 44 bp, artificial sequence
  aptamer
ctggagacgcgcagttgctacacacgcgcacatgactagaagtc

SEQ ID NO: 51 - DNA, 44 bp, artificial sequence
  aptamer
ctggagacgcgcacttcctttacatgcgcacatgactagaagtc

SEQ ID NO: 52 - DNA, 44 bp, artificial sequence
  aptamer
ctggagacgcgcagttgctttacatgcgcacatgactagaagtc

SEQ ID NO: 53 - DNA, 44 bp, artificial sequence
  aptamer
ctggagacgcgcagttgctgtacatgcgcacatgactagaagtc

SEQ ID NO: 54 - DNA, 44 bp, artificial sequence; identical to SEQ ID NO: 53
  aptamer

SEQ ID NO: 55 - DNA, 44 bp, artificial sequence
  aptamer
ctggggacgcgcagttgctgtacatgcgcacatgactagaagtc

SEQ ID NO: 56 - DNA, 44 bp, artificial sequence
  aptamer
ctggagacgcgcagttaccatacgtgcgcacatgactagaagtc

SEQ ID NO: 57 - DNA, 44 bp, artificial sequence
  aptamer
ctggagacgcgcagttgctgtacgtgcgcacatgactagaagtc

SEQ ID NO: 58 - DNA, 44 bp, artificial sequence
  aptamer
agtctccatgttgcgaaccgaatggtaaggggatcgtacactac

SEQ ID NO: 59 - DNA, 44 bp, artificial sequence; identical to SEQ ID NO: 58
  aptamer

SEQ ID NO: 60 - DNA, 44 bp, artificial sequence
  aptamer
agactccatgatgcgaaccgaatggtaagggaatcgtacaatac

SEQ ID NO: 61 - DNA, 44 bp, artificial sequence; identical to SEQ ID NO: 60
  aptamer

SEQ ID NO: 62 - DNA, 44 bp, artificial sequence
  aptamer
agactccattatgcgaaccgaatggtaagggaatcgtacaatac

SEQ ID NO: 63 - DNA, 44 bp, artificial sequence
  aptamer
agactccatgatgcgaaccgaatggtaagggaatcgtacagtac

SEQ ID NO: 64 - DNA, 44 bp, artificial sequence; identical to SEQ ID NO: 63
  aptamer

SEQ ID NO: 65 - DNA, 44 bp, artificial sequence
  aptamer
agactccatgatgcgaaccgaatggtaagggaatcgtacaatgc

SEQ ID NO: 66 - DNA, 44 bp, artificial sequence; identical to SEQ ID NO: 65
  aptamer

SEQ ID NO: 67 - DNA, 44 bp, artificial sequence; identical to SEQ ID NO: 65
  aptamer

SEQ ID NO: 68 - DNA, 44 bp, artificial sequence
  aptamer
agactccatgatgcgaaccgaatggtaagggaatcgtacgacac

SEQ ID NO: 69 - DNA, 44 bp, artificial sequence
  aptamer
agactccatggtgcgaaccgaatggtaagggaatcgtacaacac

SEQ ID NO: 70 - DNA, 44 bp, artificial sequence
  aptamer
tgactccatgatgcgaaccgaatggtaagggaatcgtacaacac

SEQ ID NO: 71 - DNA, 44 bp, artificial sequence
  aptamer
agactccatgatgcgaaccgaatggttagggaatcgtacaatac

SEQ ID NO: 72 - DNA, 44 bp, artificial sequence; identical to SEQ ID NO: 71
  aptamer

SEQ ID NO: 73 - DNA, 44 bp, artificial sequence
  aptamer
agactccatgatgcgaaccgaatggttagggaatcgtacaacac

SEQ ID NO: 74 - DNA, 44 bp, artificial sequence
  aptamer
agaccccatgatgcgaaccgaatggttagggaatcgtacagtac

SEQ ID NO: 75 - DNA, 44 bp, artificial sequence
  aptamer
agaccccctgatgcgaaccgaatggttagggaatcgtacaatac

SEQ ID NO: 76 - DNA, 44 bp, artificial sequence
  aptamer
agactccatgatgcgaaccgaatggtaagggaatcgcactgtac

SEQ ID NO: 77 - DNA, 44 bp, artificial sequence
  aptamer
agactccaagatgcgaaccgaatggtaagggaatcgtacagtac

SEQ ID NO: 78 - DNA, 44 bp, artificial sequence
  aptamer
agaccccgtgatgcgaaccgaatggtaagggaatcgcacaatac

SEQ ID NO: 79 - DNA, 44 bp, artificial sequence
  aptamer
agactccccgatgcgaaccgaatggtaagggaatcgtacgatgc

SEQ ID NO: 80 - DNA, 44 bp, artificial sequence
  aptamer
agaccccatgacgcgaaccgaatggtaagggaatcgtacaatgc

SEQ ID NO: 81 - DNA, 44 bp, artificial sequence
  aptamer
agaccccatgatgcgaaccgaatggtaagggaatcgtactacac

SEQ ID NO: 82 - DNA, 44 bp, artificial sequence
  aptamer
agaccccatgatgcgaaccgaatggtaaggggatcgtacaaccc

SEQ ID NO: 83 - DNA, 43 bp, artificial sequence
  aptamer
agactccatgatgcgaaccgaatggtaagggaatcatacaccc

SEQ ID NO: 84 - DNA, 44 bp, artificial sequence
  aptamer
agactccatgatgctaaccgaatggttagggaatcgtacgacgc

SEQ ID NO: 85 - DNA, 45 bp, artificial sequence
  aptamer
atgcagccagccgcagtgtaagtgaatgcagacatggtctaagtg

SEQ ID NO: 86 - DNA, 81 bp, artificial sequence
  aptamer
gggagatagccacgacctatgcagccagccgcagtgtaagtgaatgcagacatggtctaagtgtccaggctgtgcgaaagc

SEQ ID NO: 87 - DNA, 43 bp, artificial sequence
  aptamer
taagccgctgcgtattatagctgaatgcccctaatgggaagtg

SEQ ID NO: 88 - DNA, 44 bp, artificial sequence
  aptamer
catgccgctgcgtattatagctgaatgccccacaatgggaagtg

SEQ ID NO: 89 - DNA, 44 bp, artificial sequence
  aptamer
caagccgctgcgtattatagctgaatgccccataatgggaagtg

SEQ ID NO: 90 - DNA, 43 bp, artificial sequence
  aptamer
caagccgctgcgtatttagctgaatgccccctaatgggaagtg

SEQ ID NO: 91 - DNA, 44 bp, artificial sequence
  aptamer
ccagccgctgcgtatcatagctgaatgccccataatgggaagtg

SEQ ID NO: 92 - DNA, 43 bp, artificial sequence
  aptamer
caagccgctgcgtaatatagctgaatgccccataatggaagtg

SEQ ID NO: 93 - DNA, 44 bp, artificial sequence
  aptamer
caagcctctgcgtattatagctgaatgctccttaatggtaagtg

SEQ ID NO: 94 - DNA, 44 bp, artificial sequence
  aptamer
caagccgctgcgtattatagctgaatgctccttaatggtaagtg

SEQ ID NO: 95 - DNA, 44 bp, artificial sequence
  aptamer
ccaggcgctgcgtatcatagctgaatgctccttaacggtaagtg

SEQ ID NO: 96 - DNA, 44 bp, artificial sequence
  aptamer
caagccgctgcgttttatagctgaatgctcctcaatggtaagtg

SEQ ID NO: 97 - DNA, 44 bp, artificial sequence
  aptamer
caagccgctgcgttttatagctgaatgctccatcttggtaagtg

SEQ ID NO: 98 - DNA, 44 bp, artificial sequence
  aptamer
gtgcagccagccgcagtntaagtgagctgattgaagcatgaggg

SEQ ID NO: 99 - DNA, 45 bp, artificial sequence; identical to SEQ ID NO: 85
  aptamer

SEQ ID NO: 100 - DNA, 45 bp, artificial sequence
  aptamer
ccagcagagccgcagtttcagtgaatgcagatcatggtctaagtg

(19) United States
(12) Patent Application Publication
(10) Pub. No.: US 2012/0041056 A1
(43) Pub. Date: Feb. 16, 2012

(54) NUCLEIC ACIDS SPECIFICALLY BINDING WITH HUMAN FACTOR VII/VIIA AND USES THEREOF

(75) Inventors: Gerald Perret, Choisy Le Roi (FR); Frederic Duconge, Sceaux (FR)

(73) Assignee: LFB BIOTECHNOLOGIES, Les Ulis (FR)

(21) Appl. No.: 13/201,983

(22) PCT Filed: Feb. 19, 2010
(86) PCT No.: PCT/FR10/50291

§ 371 Date: Aug. 17, 2011

(30) Foreign Application Priority Data
Feb. 19, 2009 (FR) 0951082

(51) Int. Cl. C12N 11/10; C12N 9/96; C12N 9/64; C12Q 1/68; A61P 9/10; A61P 7/02; A61P 35/04; A61P 29/00; A61P 9/02; A61P 11/00; C07H 21/04; A61K 31/7088

(52) U.S. Cl. 514/44 R; 536/23.1; 435/188; 435/178; 435/226; 435/6.19

(57) ABSTRACT

A nucleic acid includes at least 15 nucleotides with a length specifically binding with the human factor VII/VIIa.

### FIELD OF THE INVENTION

The present invention relates to the field of the identification of ligands specific for human factor VII/VIIa, these ligands being intended in particular for purifying and detecting this protein, and also to the use thereof in the medical field.

### PRIOR ART

Factor VII (FVII) is a vitamin K-dependent glycoprotein which, in its activated form (FVIIa), participates in the coagulation process by activating factor X and factor IX in the presence of calcium and of tissue factor. FVII is secreted in the form of a single peptide chain of 406 amino acid residues, the molecular weight of which is approximately 50 kDa.

FVIIa, a vitamin K-dependent glycoprotein, therefore plays an important role in coagulation mechanisms, resulting in blood clot formation. FVIIa has the advantage of being able to act locally in the presence of the tissue factor released after tissue damage causing hemorrhages, even in the absence of factor VIII or IX. For this reason, FVIIa has been used for many years for correcting certain coagulation disorders manifested by bleeding.

FVII is used in the treatment of patients suffering from hemophilia, who have a deficiency in factor VIII (hemophilia type A) or in factor IX (hemophilia type B), and also patients who have other coagulation factor deficiencies, for example a hereditary FVII deficiency. FVII is also recommended in the treatment of strokes. It is therefore necessary to have injectable FVIIa concentrates.

The oldest method for obtaining FVIIa concentrates consisted in purifying FVIIa from plasma proteins resulting from fractionation. Document EP 0 346 241 describes, to this effect, the preparation of an FVIIa-enriched fraction obtained after adsorption and then elution of a by-product of the fractionation of plasma proteins containing FVII and FVIIa and other proteins, such as factors IX (FIX), X (FX) and II (FII), including the pre-eluate of PPSB (P=prothrombin or FII, P=proconvertin or FVII, S=Stuart factor or FX, and B=antihemophilic factor B or FIX). The drawback of this method is that the FVII obtained still contains some traces of other coagulation factors.

Likewise, document EP 0 547 932 describes a method for producing a high-purity FVIIa concentrate which is essentially free of vitamin K-dependent factors and of FVIII. The FVII obtained by this method, despite its purity, has a residual thrombogenic activity.

In the 1980s, the DNA encoding human factor VII was isolated (Hagen et al. (1986); Proc. Natl. Acad. Sci. USA; April 83 (8): 2412-6) and the corresponding protein was expressed in BHK (baby hamster kidney) mammalian cells (document EP 0 200 421). Patent application FR 06 04872 filed by the Applicant also describes the production of FVIIa in a transgenic animal.

These production methods make it possible to obtain proteins which have been made safe in terms of contamination with viruses or other pathogenic agents. Furthermore, such methods make it possible to obtain proteins of which the primary sequence, i.e. an identical linkage between the various amino acids, is identical to the human primary sequence.

However, regardless of the initial source of factor VII/VIIa, the methods implemented generate compositions enriched in human factor VII/VIIa which contain contaminating substances. For the methods for purifying factor VII/VIIa from human plasma, it would be advantageous to have a final product that is in particular free, or virtually free, of residual thrombogenic activity. For the methods for obtaining purified recombinant human factor VII/VIIa, it is essential to have a final product which is in particular free, or virtually free, of undesirable cell proteins. In particular, for the compositions of human factor VII/VIIa purified from biological fluids originating from transgenic mammals, it is important to have a final product which is free, or virtually free, of factor VII/VIIa produced, moreover, naturally by these transgenic mammals, and capable of being immunogenic in humans.

To achieve this objective, it is necessary to have efficient and specific means for purifying human factor VII/VIIa from a sample comprising said factor VII/VIIa, which can be produced easily and reproducibly.

### SUMMARY OF THE INVENTION

Single-stranded nucleic acids which are at least 15 nucleotides in length and which bind specifically to human factor VII/VIIa are provided according to the invention.

The present invention also relates to various compounds which bind specifically to human factor VII/VIIa, and which comprise in their structure at least one nucleic acid defined above.

It also relates to complexes between a nucleic acid or a compound as defined above, and (ii) a human factor VII/VIIa.

This invention also relates to a substrate for the immobilization of human factor VII/VIIa, characterized in that it comprises a solid substrate material onto which a plurality of nucleic acids or of compounds as defined above are grafted.

A subject of the invention is also a method for immobilizing human factor VII/VIIa on a substrate, comprising a step during which a sample comprising human factor VII/VIIa is brought into contact with a substrate as defined above.

The invention also relates to a method for purifying human factor VII/VIIa, comprising the following steps:
a) bringing a sample comprising human factor VII/VIIa into contact with a nucleic acid or with a substrate as defined above, in order to form a complex between (i) said nucleic acid or said substrate and (ii) the human factor VII/VIIa, and
b) releasing the human factor VII/VIIa from the complex formed in step a) and recovering the purified human factor VII/VIIa.

The invention also relates to a method for detecting the presence of human factor VII/VIIa in a sample, comprising the following steps:
a) bringing a nucleic acid or a substrate as defined above into contact with said sample; and
b) detecting the formation of complexes between (i) said nucleic acid or said substrate and (ii) the factor VII/VIIa.

The present invention also relates to preventive or curative medical uses of the nucleic acids as defined above.

### DESCRIPTION OF THE FIGURES

FIG. 1 illustrates the results of a calculation of the model for folding of a nucleic acid of the invention (Mapt2). The mFold computer program was used, according to parameters identical to those used to obtain the structure represented in FIG. 1, namely the following conditions: (i) DNA with linear sequence, (ii) folding temperature: 25° C., (iii) ionic conditions: [Na⁺]: 150 mM; [Mg⁺⁺]: 4 mM, (iv) correction type: oligomer, (v) percentage suboptimality number: 2, (vi) upper limit of the number of foldings calculated: 50, (vii) maximum distance between two base pairs: no limit, and (viii) use of the default values for the other parameters.

FIG. 2 illustrates the curves of binding of various nucleic acids of the invention (Mapt2, Mapt3 and Mapt7) to human plasma factor VII immobilized on a substrate, in a test according to the surface plasmon resonance technique. Along the X-axis: time, expressed in seconds; along the Y-axis: resonance signal, expressed in arbitrary resonance units.

FIG. 3 illustrates the curves of binding of human plasma factor VII, used at various concentrations, to a nucleic acid of the invention (Mapt2) immobilized on a substrate, in a test according to the surface plasmon resonance technique. Along the X-axis: time, expressed in seconds; along the Y-axis: resonance signal, expressed in arbitrary resonance units. The curves represent, from top to bottom of the figure, curve 1 “[FVII]”: purified human plasma FVII at the concentration of 500 nM; curve 2 “[FVII]”: purified human plasma FVII at the concentration of 250 nM; curve 3 “[FVII]”: purified human plasma FVII at the concentration of 125 nM; curve 4 “[FVII]”: purified human plasma FVII at the concentration of 62.5 nM; lower curves: preparation of purified immunoglobulins at the respective concentrations of 62, 125, 250 and 500 nM.

FIG. 4 illustrates (i) the curve of binding of recombinant human factor VII to a nucleic acid of the invention (Mapt2) immobilized on a substrate, then (ii) the curve of binding of an anti-human FVII monoclonal antibody to the immobilized nucleic acid/FVII complex formed in (i), in a test according to the surface plasmon resonance technique. Along the X-axis: time, expressed in seconds; along the Y-axis: resonance signal, expressed in arbitrary resonance units.

FIG. 5 illustrates a curve of saturation of a substrate, on which a nucleic acid of the invention (Mapt2) is immobilized, with human plasma factor VII injected continuously over time. Test according to the surface plasmon resonance technique. Along the X-axis: time, expressed in seconds; along the Y-axis: resonance signal, expressed in arbitrary resonance units. Top curve: signal obtained with purified human FVII at the concentration 500 nM.

FIG. 6 illustrates the curves of the kinetics of binding of human plasma factor VII to a nucleic acid of the invention (Mapt2) immobilized on a substrate, in a test according to the surface plasmon resonance technique. Along the X-axis: time, expressed in seconds; along the Y-axis: resonance signal, expressed in arbitrary resonance units.

FIG. 7 illustrates the curves of binding of factor VII proteins of various origins to a nucleic acid of the invention (Mapt2) immobilized on a substrate, in a test according to the surface plasmon resonance technique. Along the X-axis: time, expressed in seconds; along the Y-axis: resonance signal, expressed in arbitrary resonance units.

FIG. 8 illustrates the curves of binding of human plasma factor VII and of rabbit recombinant factor VII to a nucleic acid of the invention (Mapt2) immobilized on a substrate, in a test according to the surface plasmon resonance technique. Along the X-axis: time, expressed in seconds; along the Y-axis: resonance signal, expressed in arbitrary resonance units.

FIG. 9 illustrates the structure of a compound for specific binding to human factor VII/VIIa, comprising an aptamer according to the invention (Mapt2) coupled to a PEG spacer chain, the spacer chain being itself coupled to a biotin molecule.

FIG. 10 illustrates an alignment of various aptamers which bind specifically to human factor VII/VIIa, selected at the end of a cycle of implementation of a process of SELEX type. The sequences represented in FIG. 10 are, from top to bottom of the figure, respectively the sequences SEQ ID No. 87 to SEQ ID No. 100.

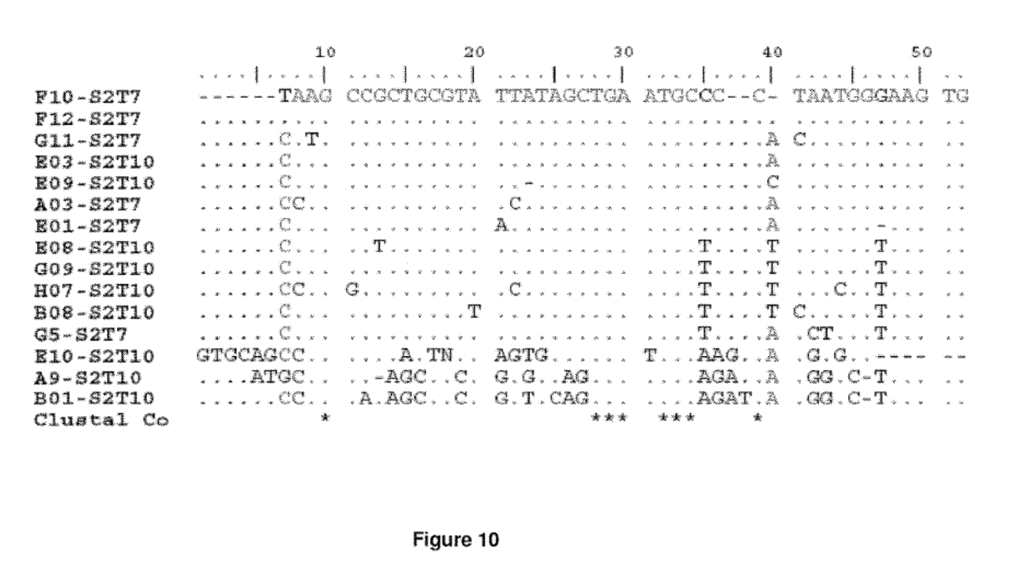

FIG. 11 illustrates a chromatographic profile obtained during the implementation of the method for purifying a recombinant human factor VII produced in rabbit milk, with the affinity substrate in which anti-human FVII nucleic aptamers are immobilized. Along the X-axis: time; along the Y-axis: the value of absorbance (OD) at 254 nanometers.

FIG. 12 illustrates the curves of binding on a series of 27 nucleic aptamers of the invention to human plasma factor VII immobilized on a substrate, in a test according to the surface plasmon resonance technique. Along the X-axis: time, expressed in seconds; along the Y-axis: resonance signal, expressed in arbitrary resonance units.

FIG. 13 illustrates the individual values of the signal for stable binding of each of the 27 aptamers tested. Along the X-axis: each of the 27 aptamers tested; along the Y-axis: resonance signal, expressed in arbitrary resonance units.

FIG. 14 represents the curve of the values of the measurement of absorbance at 280 nm as a function of time.

FIG. 15 represents the image of an SDS PAGE electrophoresis gel in which lane No. 1 corresponds to the starting-product fraction and lane No. 2 to the elution fraction.

FIG. 16 represents the curves of binding of the immobilized aptamer Mapt2 to recombinant human factor VII produced in the milk of a transgenic rabbit. The arrows correspond to the time of the various injections, respectively from left to right in FIG. 16: 1: injection of recombinant factor VII; 2: injection of a buffer containing 1M NaCl; 3: injection of a buffer containing 2M NaCl; 4: injection of a buffer containing 3M NaCl; 5: injection of a 50 mM Tris, 10 mM EDTA buffer. Along the X-axis: time, expressed in seconds; along the Y-axis: the value of the response signal, expressed in arbitrary units (RU).

FIG. 17 represents the curves of binding of the immobilized aptamer Mapt2 to recombinant human factor VII produced in the milk of a transgenic rabbit. The arrows correspond to the time of the various injections, respectively from left to right in FIG. 17: 1: 50% propylene glycol; 2: 10 mM EDTA.

### DETAILED DESCRIPTION OF THE INVENTION

The invention provides novel means capable of binding specifically to human factor VII/VIIa, which are small in size and easy and inexpensive to synthesize, and which can be used in all the fields of application in which such means are used, including for purifying human factor VII/VIIa, for detecting human factor VII/VIIa and for use as an active ingredient of medicaments intended for preventing or treating coagulation disorders.

More specifically, the Applicant has constructed a family of single-stranded nucleic acids capable of binding specifically to human factor VII/VIIa, which have numerous structural characteristics, which will be detailed later in the present description, in common.

As will be detailed later, the family of nucleic acids of the invention, which are capable of binding specifically to human factor VII/VIIa, consists of single-stranded nucleic acids, preferentially of DNA type, which, owing to certain structural characteristics provided by their nucleotide sequence, are capable of adopting spatial conformations which contribute to imparting to said nucleic acids their abovementioned properties of binding to factor VII/VIIa. Generically, the nucleic acids of the invention can also be called nucleotide “aptamers”, with reference to a term commonly used by those skilled in the art to denote molecules of this type.

Nucleic aptamers capable of binding to various proteins involved in the blood coagulation pathway are already known in the prior art, including aptamers which bind von Willebrand factor (PCT application No. WO 2008/150495), aptamers which bind alpha-thrombin (European patent application No. EP 1 972 693) or thrombin (Zhao et al., 2008, Anal Chem, Vol. 80(19): 7586-7593), aptamers which bind factor IX/IXa (Subash et al., 2006, Thromb Haemost, Vol. 95: 767-771; Howard et al., 2007, Atherioscl Thromb Vasc Biol, Vol. 27: 722-727; PCT application No. WO 2002/096926; U.S. Pat. No. 7,312,325), and aptamers which bind factor X/Xa (PCT application No. WO 2002/096926; U.S. Pat. No. 7,312,325).

Nucleic aptamers which bind to human factor VII/VIIa have also been described in the prior art (Rusconi et al., 2000, Thromb Haemost, Vol. 84(5): 841-848; Layzer et al., 2007, Spring, Vol. 17: 1-11).

A subject of the present invention is a nucleic acid which is at least 15 nucleotides in length and which binds specifically to human factor VII/VIIa.

In the present description, a single-stranded nucleic acid which binds specifically to human factor VII/VIIa can also be denoted “nucleic aptamer”, “aptamer”, “aptamer which binds to human factor VII/VIIa” or else “anti-human FVII/VIIa aptamer”.

The term “human factor VII/VIIa” encompasses a human factor VII/VIIa of natural origin and a recombinant human factor VII/VIIa. For the purpose of the present description, a human factor VII/VIIa is considered with reference to its amino acid sequence, i.e. independently of the fact that the protein is glycosylated or nonglycosylated and, if the protein is glycosylated, irrespective of the type of glycosylation.

As will also be illustrated in greater detail subsequently, certain embodiments of the nucleic acids which bind specifically to human factor VII/VIIa of the invention have various common structural characteristics, including therein a sequence comprising, from the 5′ end to the 3′ end, successively (i) an invariable specific nucleotide sequence of approximately 20 nucleotides in length, followed by (ii) a variable nucleotide sequence approximately 40 to 50 nucleotides in length, followed by (iii) an invariable specific nucleotide sequence approximately 20 nucleotides in length. It is specified that the variable nucleotide sequences (ii) can have, with respect to one another, a very strong nucleotide sequence identity.

The Applicant has therefore constructed a family of nucleic aptamers which bind specifically to human factor VII/VIIa, of which it has been able to show the existence of relationships between (i) the common structural characteristics and (ii) the common functional characteristic(s).

From a structural point of view, the family of nucleic acids, or nucleic aptamers, which bind specifically to human factor VII/VIIa, of the invention, comprise at least 15 consecutive nucleotides of a polynucleotide having at least 40% nucleotide identity with the nucleic acid of formula (I) below:

5′-[SEQ ID No. 1]x-[SEQ ID No. X]-[SEQ ID No. 2]y-3′ (I),

in which:
“SEQ ID No. X” consists of a nucleic acid chosen from the group consisting of nucleic acids of sequences SEQ ID No. 3 to SEQ ID No. 85 and SEQ ID No. 87 to SEQ ID No. 100,
“x” is an integer equal to 0 or 1, and
“y” is an integer equal to 0 or 1.

In some embodiments, the acid of sequence SEQ ID No. X has a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

In other embodiments, the nucleic acid of sequence SEQ ID No. X has a length of 43, 44, 45, 46, 47, 48 or 49 nucleotides.

In some other embodiments which are preferred, the nucleic acid of sequence SEQ ID No. X has a length of 43, 44 or 45 nucleotides.

As already mentioned above, the nucleic acid of formula (I) is at least 15 nucleotides in length.

In some embodiments, the nucleic acid of formula (I) is at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or 81 nucleotides in length, which encompasses the nucleic acids having exactly each of the lengths specified.

In some embodiments of the method for obtaining the aptamers of formula (I), the successive selection cycles carried out in order to construct the family of nucleic acids of interest which bind specifically to human factor VII/VIIa have resulted in the isolation and characterization, at each successive selection step, of sets and subsets of nucleic aptamers comprising, at their 5′ and 3′ ends, respectively, the sequences SEQ ID No. 1 and SEQ ID No. 2, structurally framing a variable sequence SEQ ID No. X. In the main family of nucleic aptamers of the invention, all of the variable sequences SEQ ID No. X have, with respect to one another, a nucleotide sequence identity of at least 40%. This means that, for the sequence SEQ ID No. X, the structural constraints for retaining the property of binding to human factor VII/VIIa are much less that the structural constraints for the sequences located, respectively, at the 5′ and 3′ ends of these nucleic aptamers.

When the integer “x” is equal to 0 and the integer “y” is equal to 1, the nucleic aptamers of the invention encompass the nucleic acids comprising at least 15 consecutive nucleotides of a polynucleotide having at least 40% nucleotide identity with the nucleic acid of formula (I-1) below:

5′-[SEQ ID No. X]-[SEQ ID No. 2]-3′ (I-1).

When the integer “x” is equal to 1 and the integer “y” is equal to 0, the nucleic aptamers of the invention encompass the nucleic acids comprising at least 15 consecutive nucleotides of a polynucleotide having at least 40% nucleotide identity with the nucleic acid of formula (I-2) below:

5′-[SEQ ID No. 1]-[SEQ ID No. X]-3′ (I-2).

When the integer “x” is equal to 0 and the integer “y” is equal to 0, the nucleic aptamers of the invention encompass the nucleic acids comprising at least 15 consecutive nucleotides of a polynucleotide having at least 40% nucleotide identity with the nucleic acid of formula (I-3) below:

5′-[SEQ ID No. X]-3′ (I-3).

The nucleic aptamers above therefore encompass the nucleic acids comprising at least 15 consecutive nucleotides of a polynucleotide having at least 40% nucleotide identity with a nucleic acid chosen from the group consisting of the nucleic acids of sequences SEQ ID No. 3 to SEQ ID No. 85 and SEQ ID No. 87 to SEQ ID No. 100.

Generally, a first nucleotide having at least 40% nucleotide identity with a second polynucleotide or nucleic acid has at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 100% nucleotide identity with said second polynucleotide or nucleic acid.

In some embodiments of a nucleic acid of the invention comprising the sequence SEQ ID No. X, said sequence SEQ ID No. X is chosen from the group consisting of the nucleic acids having at least 15 consecutive nucleotides of a sequence having at least 40% nucleotide identity with at least one of the sequences SEQ ID No. 3 to SEQ ID No. 85 and SEQ ID No. 87 to SEQ ID No. 100.

In some embodiments of a nucleic acid of the invention comprising the sequence SEQ ID No. X, said sequence SEQ ID No. X is chosen from the group consisting of the nucleic acids having at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 100% nucleotide identity with at least one of the sequences SEQ ID No. 3 to SEQ ID No. 85 and SEQ ID No. 87 to SEQ ID No. 100.

It results from the aforementioned that the present invention encompasses a family of single-stranded nucleic acids having at least 15 consecutive nucleotides of the series of formula (I) defined above.

For the purpose of the present invention, the “percentage identity” between two nucleic acid sequences is determined by comparing the two sequences aligned in an optimal manner, through a window of comparison.

The part of the nucleotide sequence that is in the window of comparison can thus comprise additions or deletions (for example gaps) compared with the reference sequence (which does not comprise these additions or deletions) in such a way as to obtain optimal alignment between the two sequences.

The percentage identity is calculated by determining the number of positions at which an identical nucleic base is observed for the two sequences compared, then in dividing the number of positions at which there is identity between the two nucleic bases by the total number of positions in the window of comparison, and then by multiplying the result by one hundred in order to obtain the percentage nucleotide identity of the two sequences with respect to one another.

The optimal alignment of the sequences for the comparison can be carried out by computer using known algorithms.

Entirely preferably, the percentage sequence identity is determined using the CLUSTAL W software (version 1.82), the parameters being set as follows: (1) CPU MODE=ClustalW mp; (2) ALIGNMENT=“full”; (3) OUTPUT FORMAT=“aln w/numbers”; (4) OUTPUT ORDER=“aligned”; (5) COLOR ALIGNMENT=“no”; (6) KTUP (word size)=“default”; (7) WINDOW LENGTH=“default”; (8) SCORE TYPE=“percent”; (9) TOPDIAG=“default”; (10) PAIRGAP=“default”; (11) PHYLOGENETIC TREE/TREE TYPE=“none”; (12) MATRIX=“default”; (13) GAP OPEN=“default”; (14) END GAPS=“default”; (15) GAP EXTENSION=“default”; (16) GAP DISTANCES=“default”; (17) TREE TYPE=“cladogram” and (18) TREE GRAPH DISTANCES=“hide”.

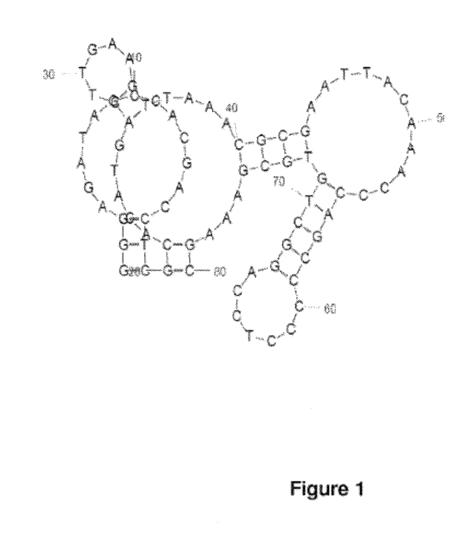

On the basis of the structure of a nucleic acid of the invention which is represented in FIG. 1, those skilled in the art can easily determine the possible specific sequences for the sequence SEQ ID No. X, within the collection of the finite number of possible series of nucleotides.

By way of illustration and for some embodiments of a nucleic aptamer of the invention, those skilled in the art can easily, on the basis of the above structural definition of the nucleic aptamers of formula (I), automatically generate, for example by means of a digital computer, the memory of which is loaded with a suitable set of instructions, all the possible sequences SEQ ID No. X. Where appropriate, those skilled in the art can then automatically determine, by means of said digital computer, respectively (i) those of the sequences of which the spatial structure model is similar or identical to that of the nucleic aptamer of FIG. 1, and (ii) those of which the structure of the nucleic aptamer is different from that of the nucleic aptamer of FIG. 1.

The nucleic aptamers which have a spatial structure similar or identical to that of the nucleic aptamer of FIG. 1 encompass those which comprise the series of loops and stems which has previously been described for this nucleic aptamer.

In order to determine the spatial structure of a nucleic acid of formula (I), those skilled in the art can in particular, on the basis of the description of its nucleotide sequence, generate a structural model using the mFold® computer program described by Zuker (2003, Nucleic Acids Research, Vol. 231(13): 3406-3413), and which can also be used at the following web address: http://mfold.bioinfo.rpi.edu/.

Preferentially, the mFold computer program is used according to parameters identical to those used to obtain the structure represented in FIG. 1, namely the following conditions: (i) DNA with linear sequence, (ii) folding temperature: 25° C., (iii) ionic conditions: [Na⁺]: 150 mM; [Mg⁺⁺]: 4 mM, (iv) correction type: oligomer, (v) percentage suboptimality number: 2, (vi) upper limit of the number of foldings calculated: 50, (vii) maximum distance between two base pairs: no limit, and (viii) use of the default values for the other parameters.

Those skilled in the art then carry out a step of comparison between (i) the model of the structure of the aptamer of FIG. 1 and (ii) the model of the structure of the aptamer of formula (I) which has just been generated, and they positively select the aptamer of formula (I) which has just been generated if its structural model is identical or similar to that of the aptamer represented in FIG. 1.

In any event, in order to confirm the positive selection of the newly generated aptamer of formula (I), those skilled in the art can verify its human factor VII/VIIa-binding properties, for example according to one of the methods specified in the present description, in particular in the examples.

In some preferred embodiments of a nucleic aptamer of the invention, said nucleic aptamer comprises at least 15 consecutive nucleotides of a polynucleotide having at least 80% nucleotide identity with the nucleic acid of formula (I), which encompasses the aptamers comprising 15 consecutive nucleotides of a polynucleotide having at least 81%, 82%, 83%, 84%, 85%, 86%, 87%, 88%, 89%, 90%, 91%, 92%, 93%, 94%, 95%, 96%, 97%, 98% or 100% nucleotide identity with said nucleic acid of formula (I).

In preferred embodiments of a nucleic acid of formula (I), the sequence SEQ ID No. X has at least 80% nucleotide identity with at least one of the sequences SEQ ID No. 3 to SEQ ID No. 85 and SEQ ID No. 87 to SEQ ID No. 100, which encompasses the sequences having at least 81%, 82%, 83%, 84%, 85%, 86%, 87%, 88%, 89%, 90%, 91%, 92%, 93%, 94%, 95%, 96%, 97%, 98% or 100% nucleotide identity with at least one of the sequences SEQ ID No. 3 to SEQ ID No. 85 and SEQ ID No. 87 to SEQ ID No. 100.

In other preferred embodiments of a nucleic acid of formula (I), the sequence SEQ ID No. X is chosen from the group consisting of the sequences SEQ ID No. 3 to SEQ ID No. 85 and SEQ ID No. 87 to SEQ ID No. 100.

In yet other preferred embodiments of a nucleic acid of formula (I), the sequence SEQ ID No. X is chosen from the group consisting of the sequences SEQ ID Nos. 3, 5, 6, 10, 11, 14, 15, 16, 17, 19, 20, 23, 24, 25, 27, 28, 29, 30, 32, 33, 34, 35, 36, 37, 38, 39 and 40.

According to the invention, a nucleic acid which binds to human factor VII/VIIa consists of a single-stranded nucleic acid capable of forming a complex with human factor VII/VIIa, when it is brought into contact with the latter.

The nucleic acids which bind to human factor VII/VIIa therefore encompass those for which complexes with human factor VII/VIIa can be detected after a prior step of bringing said respectively nucleic and protein partners into contact.

The detection of complexes formed with a nucleic acid which binds to human factor VII/VIIa can be easily carried out by those skilled in the art, for example by implementing a surface plasmon resonance detection technique, including the Biacore® technique, as is illustrated in the examples. Those skilled in the art can also easily detect the formation of complexes between a nucleic acid of interest and human factor VII/VIIa by means of conventional techniques of the ELISA type, as is also illustrated in the examples.

As is illustrated in the examples, a nucleic acid of formula (I) has a strong capacity for binding to any type of human factor VII/VIIa. In particular, a nucleic acid of formula (I) binds both to natural human factor VII/VIIa and to recombinant human factor VII/VIIa.

Without wishing to be bound by any theory, the applicant thinks that the results presented in the examples show that a nucleic acid of formula (I) according to the invention has a strong capacity to bind to human factors VII/VIIa having distinct types of glycosylation. In other words, the applicant thinks that a nucleic acid of formula (I) is capable of effectively binding not only to factor VII/VIIa originating from natural sources, including human plasma, but also to a recombinant human factor VII/VIIa produced in a transgenic animal, preferentially a transgenic mammal, of various species, including rabbit, of which the types of glycosylation can differ, even slightly, from the type of glycosylation of the human factor VII/VIIa produced naturally in blood plasma.

According to the invention, a nucleic acid which binds “specifically” to human factor VII/VIIa consists of a nucleic acid which has a capacity to bind to human factor VII/VIIa that is greater than its capacity to bind to any other protein, including to factors VII/VIIa encoded by the genome of a nonhuman mammal, such as rabbit factor VII/VIIa.

For the purpose of the present description, a first nucleic acid has a capacity to bind to human factor VII/VIIa that is greater than that of a second nucleic acid when, using any one of the above techniques for detecting binding, and under the same test conditions, a statistically significant higher binding signal value is obtained with the first nucleic acid, compared with that obtained with the second nucleic acids. By way of illustration, when the technique for detecting binding that is used is the Biacore® technique, as in the examples, a first nucleic acid has a capacity to bind to human factor VII/VIIa that is greater than that of a second nucleic acid, when the resonance signal value for the first nucleic acid, regardless of that measurement unit expressed, is statistically greater than the resonance signal value measured for the second nucleic acid. Two “statistically” distinct measurement values encompass two values which have, between them, a difference greater than the measurement error of the technique for detecting binding that is used.

As is illustrated in the examples, the capacity of the nucleic acids of the invention to bind specifically to human factor VII/VIIa was tested.

Generally, a nucleic acid of formula (I) has a dissociation constant value for human factor VII/VIIa of at most 500 nM, better still of at most 200 nM.

It has been shown that the nucleic acids of formula (I) have a capacity to bind to human factor VII/VIIa which is significantly greater than their capacity to bind to any factor VII/VIIa originating from a nonhuman mammal. In particular, although a nucleic acid of formula (I) has a strong capacity to bind to any type of human factor VII/VIIa, including natural or recombinant, it has a weak or zero capacity to bind to a factor VII/VIIa encoded by the genome of a nonhuman mammal, including a rabbit factor VII/VIIa.

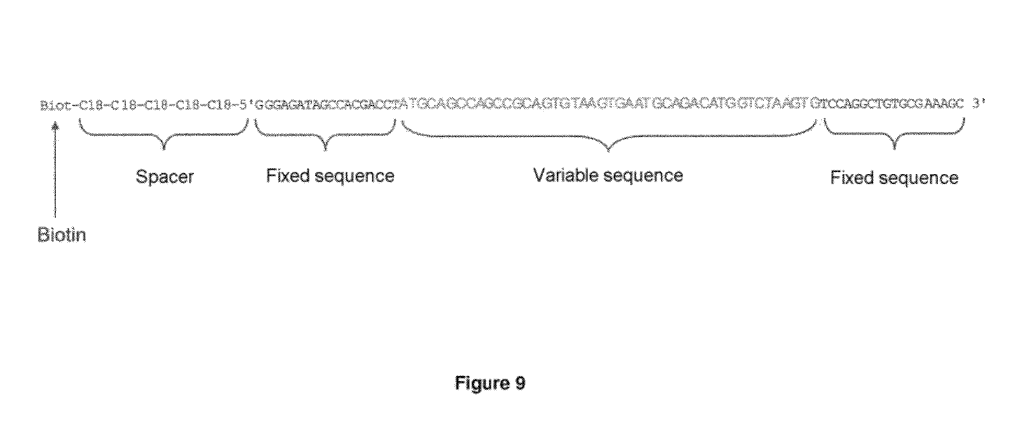

This advantageous characteristic of a nucleic acid of formula (I) is illustrated in the examples, in particular with the nucleic acid of sequence SEQ ID No. 86, which consists of a nucleic acid of formula (I) in which the sequence SEQ ID No. X consists of the sequence SEQ ID No. 85 and the structure of which, after coupling thereof with PEG and biotin, is represented in FIG. 9.

Thus, for the nucleic acid of formula (I) of sequence SEQ ID No. 86, it has been determined, according to the Biacore® technique, that the value for the capacity for binding to human factor VII/Vila, expressed as the dissociation constant Kd, is approximately 100 nM. Furthermore, said nucleic acid of formula (I) has a capacity for binding both to human plasma factor VII/VIIa and to recombinant human factor VII/VIIa, for example produced in a transgenic rabbit, which is of the same order of magnitude.

It has also been shown in the examples that the complexes between a nucleic acid of formula (I) and a human factor VII/VIIa are stoichiometric, i.e. the ratio of the number of molecules of nucleic acid of formula (I) to the number of molecules of human factor VII/VIIa that are complexed is approximately 1:1, and can more particularly be 1:1.

Thus, according to another aspect, the capacity of a nucleic acid of formula (I) to bind “specifically” to human factor VII/VIIa can also be expressed as the ratio of the dissociation constants Kd, respectively for human factor VII/VIIa and a nonhuman factor VII/VIIa.

According to yet another characteristic of a nucleic acid of formula (I) according to the invention, the capacity of said nucleic acid to bind specifically to human factor VII/VIIa can also be expressed by the following condition (A):

human Kd/nonhuman Kd<0.01 (A),

in which:
“human Kd” is the dissociation constant of a nucleic acid of formula (I) for human factor VII/VIIa, expressed in molar units, and
“nonhuman Kd” is the dissociation constant of said nucleic acid of formula (I) for nonhuman factor VII/VIIa, expressed in the same molar units.

Thus, for a nucleic acid which binds specifically to human factor VII/VIIa according to the invention, the human Kd/nonhuman Kd ratio is preferentially less than 0.01, better still less than 0.001.

These characteristics of specificity of the binding of a nucleic acid of formula (I) according to the invention to human factor VII/VIIa illustrate that the aptamer nucleic acids of the invention can be advantageously used for distinguishing between a human factor VII/VIIa and a factor VII/VIIa originating from a nonhuman mammal, for example a rabbit factor VII/VIIa.

In particular, a nucleic acid of formula (I) according to the invention can advantageously be used in a means for purifying a human factor VII/VIIa, including from a complex starting material capable of containing a factor VII/VIIa originating from a nonhuman mammal. In particular, a nucleic acid of formula (I) can be used in a means for purifying recombinant factor VII/VIIa in a biological fluid from a rabbit that is transgenic for human factor VII/VIIa, said biological fluid being capable of containing a factor VII/VIIa produced naturally by the rabbit.

As is illustrated in the examples, another characteristic of a nucleic acid of formula (I) is its ability, in a complex with human factor VII/VIIa, to release the human factor VII/VIIa by incubation of said complex with a metal-cation-chelating agent. It has in particular been shown that the molecules of factor VII/VIIa complexed with a nucleic acid of formula (I) are released from said complexes by bringing into contact with a metal-cation-chelating agent such as EDTA.

Thus, according to yet another aspect, the binding of a nucleic acid of formula (I) according to the invention to human factor VII/VIIa can be dissociated by bringing into contact with a metal-cation-chelating agent, including by bringing into contact with EDTA.

This additional characteristic of a nucleic acid of formula (I) according to the invention is particularly advantageous for the use of said nucleic acid in a means for purifying human factor VII/VIIa. Specifically, in such an application for purification, the human factor VII/VIIa, immobilized in a complex with a nucleic acid of formula (I), can then be recovered in purified form by simply bringing into contact or incubating with a metal-cation-chelating agent, therefore without the need to use substances known to at least partially denature human factor VII/VIIa, such as acidic conditions or urea.

For its use in a means for purifying human factor VII/VIIa, a nucleic acid of formula (I) according to the invention is preferentially immobilized on a solid substrate. Said solid substrate encompasses solid particles, chromatography substrates, etc. The techniques for immobilizing nucleic acids of interest on very varied types of solid substrates are well known to those skilled in the art.

The solid substrate may be an affinity chromatography column composed of a gel derived from agarose or from cellulose or of a synthetic gel such as an acrylamide, methacrylate or polystyrene derivative, or a chip such as a chip suitable for surface plasmon resonance, a membrane such as a polyamide, polyacrylonitrile or polyester membrane, or else a magnetic or paramagnetic bead.

For its use in a means for purifying human factor VII/VIIa, a nucleic acid of formula (I) according to the invention is preferably included in a chemical structure which also comprises a spacer means and, where appropriate, a means for immobilization on a solid substrate.

Thus, the present invention also relates to a compound which binds specifically to human factor VII/VIIa, characterized in that it is of formula (II) below:

[SPAC]-[NUCL] (II), in which:
[SPAC] signifies a spacer chain, and
[NUCL] signifies a nucleic acid which binds specifically to human factor VII/VIIa, comprising at least 15 consecutive nucleotides of a polynucleotide having at least 40% nucleotide identity with the nucleic acid of formula (I).

The compound above constitutes a particular embodiment of a means for purifying human factor VII/VIIa.

The “spacer chain”, denoted [SPAC] in the compound of formula (II), may be of any known type. The function of said spacer chain is to physically distance the nucleic acid [NUCL] from the surface of the solid substrate on which said compound can be immobilized and to allow a relative mobility of the nucleic acid [NUCL], relative to the surface of the solid substrate on which it can be immobilized. The spacer chain limits or prevents steric hindrances, owing to the solid substrate being too close to the nucleic acid of formula (I), from impeding the binding events between said nucleic acid and molecules of human factor VII/VIIa that may be brought into contact therewith.

In the compound of formula (II), the spacer chain is preferentially bonded to the 5′ end or to the 3′ end of the nucleic acid [NUCL].

Advantageously, the spacer chain is bonded both to one end of the aptamer and to the solid substrate. This construction with a spacer has the advantage of not directly immobilizing the aptamer on the solid substrate. Preferably, the spacer chain is a nonspecific oligonucleotide or polyethylene glycol (PEG). When the spacer chain consists of a nonspecific oligonucleotide, said oligonucleotide advantageously comprises at least 5 nucleotides in length, preferably between 5 and 15 nucleotides in length.

In the embodiments of a compound of formula (II) in which the spacer chain consists of a polyethylene glycol, said spacer chain encompasses a polyethylene glycol of PEG(C18) type.

In order to immobilize the aptamer directly on the solid substrate, or on the spacer chain, the nucleic acid [NUCL] can be chemically modified with various chemical groups, such as groups for immobilizing said nucleic acid covalently, for instance thiols, amines or any other group capable of reacting with chemical groups present on the solid substrate. In some embodiments, the spacer chain is itself able to be immobilized on the solid substrate, where appropriate after modification of said spacer chain with one or more suitable chemical groups. In yet other embodiments, the spacer chain is bonded to a compound which allows the immobilization of the compound of interest comprising the nucleic aptamer on the mobile substrate.

Thus, the present invention also relates to a compound which binds specifically to human factor VII/VIIa, characterized in that it is of formula (III) below:

[FIX]-[SPAC]-[NUCL] (III), in which:
[FIX] signifies a compound for immobilization on a substrate,
[SPAC] signifies a spacer chain, and
[NUCL] signifies a nucleic acid which binds specifically to human factor VII/VIIa comprising at least 15 consecutive nucleotides of a polynucleotide having at least 40% nucleotide identity with the nucleic acid of formula (I).

The compound of formula (III) above constitutes an embodiment of a means for purifying human factor VII/VIIa.

Said means for purifying human factor VII/VIIa, of formula (II) or of formula (III), is preferentially in a form bonded to a solid substrate.

In the compound of formula (III), the compound [FIX] consists of a compound chosen from (i) a compound capable of forming one or more covalent bonds with the surface material of a solid substrate and (ii) a compound capable of binding specifically on the solid substrate by means of weak noncovalent bonds, including hydrogen bonds, electrostatic forces or Van der Waals forces.

The first type of compound [FIX] encompasses bifunctional coupling agents, such as glutaraldehyde, STAB or else SMCC.

The compound SIAB, described by Hermanson G. T. (1996, Bioconjugate techniques, San Diego: Academic Press, pp 239-242), is the compound of formula (I) below:

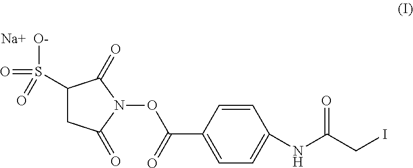

The compound STAB comprises two reactive groups, respectively an iodoacetate group and a sulfo-NHS ester group, these groups reacting respectively with amino and sulfhydryl groups.

The compound SMCC, which is described by Samoszuk M. K. et al. (1989, Antibody, Immunoconjugates Radiopharm., 2(1): 37-46), is the compound of formula (II) below:

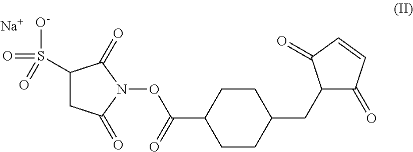

The compound SMCC comprises two reactive groups, respectively a sulfo-NHS ester group and a maleimide group, which react respectively with an amino group and a sulfhydryl group.

The second type of compound [FIX] encompasses biotin, which is capable of binding specifically, in a noncovalent manner, to avidin or streptavidin molecules present on the solid substrate.

In some embodiments, once immobilized on the solid substrate via the spacer chain, the aptamer is advantageously modified and its free end (end not bonded to the spacer) by virtue of, and without being limited thereto, a chemically modified nucleotide (such as 2′-O-methyl- or 2′-fluoropyrimidine, 2′-ribopurine, phosphoramidite), an inverted nucleotide or a chemical group (PEG, polycations, cholesterol). These modifications make it possible to protect certain aptamers against enzymatic degradations.

The solid substrate may be an affinity chromatography column composed of a gel derived from agarose or from cellulose or of a synthetic gel such as an acrylamide, methacrylate or polystyrene derivative, a chip such as a chip suitable for surface plasmon resonance, a membrane such as a polyamide, polyacrylonitrile or polyester membrane, or a magnetic or paramagnetic bead.

The present invention also relates to a complex between:
(i) a substance chosen from a nucleic acid of formula (I), a compound of formula (II) and a compound of formula (III), and
(ii) a human factor VII/VIIa.

A subject of the present invention is also a substrate for the immobilization of human factor VII/VIIa, characterized in that it comprises a solid substrate material onto which a plurality of molecules each consisting of, or each comprising, a nucleic aptamer are grafted, said molecules being chosen from (a) a nucleic acid of formula (I), (b) a compound of formula (II) and (c) a compound of formula (III).

The above substrate can be used practically in all the applications for which the intention is to immobilize human factor VII/VIIa, which encompasses applications for the purposes of purifying factor VII/VIIa and applications for the purposes of detection human factor VII/VIIa.

The preparation of substrates on which nucleic aptamers of the invention which bind specifically to human factor VII/VIIa are immobilized is widely illustrated in the examples, in which the aptamers of the invention are used in particular as agents for capturing human FVII/VIIa, that can be used for purifying or for detecting human FVII/VIIa in samples.

The present invention therefore also relates to a method for immobilizing human factor VII/VIIa on a substrate, comprising a step during which a sample comprising human factor VII/VIIa is brought into contact with a solid substrate on which a substance chosen from a nucleic acid of formula (I), a compound of formula (II) and a compound of formula (III) has previously been immobilized. Said method may comprise, depending on the technical objectives pursued, an additional step of recovering the immobilized molecules of human factor VII/VIIa, complexed with the molecules of nucleic acids of formula (I). The additional step of recovering the factor VII/VIIa preferentially consists of a step of bringing the complexes of factor VII/VIIa with the nucleic acids of formula (I) into contact with a metal-cation-chelating agent, such as EDTA.

A subject of the present invention is therefore a method for purifying human factor VII/VIIa, comprising the following steps:
a) bringing a sample comprising human factor VII/VIIa into contact with a nucleic acid of formula (I), a compound of formula (II) or a compound of formula (III) or with a solid substrate as defined in the present description, in order to form a complex between (i) said nucleic acid or said substrate and (ii) the human factor VII/VIIa, and
b) releasing the human factor VII/VIIa from the complex formed in step a) and recovering the purified human factor VII/VIIa.

For the implementation of the methods of protein purification by affinity chromatography using solid substrates on which the aptamers of interest are immobilized, those skilled in the art may refer to the studies described by Romig et al. (1999, J Chomatogr B Biomed Sci Apl, Vol. 731(2): 275-284).

It has been shown in the examples that the factor VII capture conditions are improved when a buffer with a low MgCl₂ concentration or even a buffer free of MgCl₂ is used in step a).

The expression “buffer with a low MgCl₂ concentration” is intended to mean, according to the invention, a buffer of which the final MgCl₂ concentration is less than 1 mM.

A buffer of which the MgCl₂ concentration is less than 1 mM encompasses the buffers of which the MgCl₂ concentration is less than 0.5 mM, 0.1 mM, 0.05 mM and 0.01 mM, advantageously equal to 0 mM.

In one particular embodiment, the method comprises a step a′), step a′ following step a) and preceding step b), which consists of a step of washing the affinity substrate with a washing buffer. Advantageously, the method comprises a step a′) of washing the affinity substrate during which the ionic strength is increased, i.e. use is made of a washing buffer of which the ionic strength is increased compared with the ionic strength of the buffer used in step a). Advantageously, the ionic strength of the washing buffer used in step a′) is 2- to 500-fold greater than the ionic strength of the buffer used in step a). Advantageously, the ionic strength of the washing buffer is 100- to 500-fold greater, preferably 200- to 500-fold greater, than the ionic strength of the buffer used in step a).

It has been shown in the examples that the use, in washing step a′), of a washing buffer having a high ionic strength, in particular a buffer having a high NaCl concentration, makes it possible to effectively eliminate the substances bound nonspecifically to the affinity substrate without simultaneously affecting, in a detectable manner, the binding of factor VII to the affinity substrate.

A washing buffer having a final NaCl concentration of at least 1 M is thus preferably used in step a′).

According to the invention, a washing buffer having a final NaCl concentration of at least 1 M encompasses the washing buffers having a final NaCl concentration of at least 1.5 M, 2 M, 2.5 M or at least 3 M.

Preferably, a washing buffer used in step a′) of the method has a final NaCl concentration of at most 3.5 M. Advantageously, a washing buffer used in step a′) of the method has a final NaCl concentration of between 1.5 and 3.5, preferably between 2 and 3.5, including preferably between 2.5 and 3.5, for example preferably between 3 and 3.5.

It has also been shown in the examples that the use, in step a′), of a washing buffer having a high hydrophobicity, in particular a high propylene glycol concentration, makes it possible to effectively eliminate the substances bound nonspecifically to the affinity substrate without simultaneously affecting, in a detectable manner, the binding of factor VII to the affinity substrate.

A washing buffer having a final propylene glycol content of at least 20% (v/v) is thus preferably used in step a′).

According to the invention, a washing buffer having a final propylene glycol content of at least 20% encompasses the washing buffers having a final propylene glycol content of at least 25%, 30%, 35%, 40%, 45%, 50%, 55%, or at least 60% by volume, relative to the total volume of the washing buffer.

Preferably, a washing buffer used in step a′) of the method has a final propylene glycol content of at most 50%. Advantageously, a washing buffer used in step a′) of the method has a final propylene glycol content of between 20% and 50%, preferably between 30% and 50%.

According to one particular embodiment, the washing buffer used in step a′) contains both NaCl and propylene glycol as described above.

In addition, in some embodiments of the purification method above, step b) is carried out by bringing the affinity substrate into contact with an elution buffer containing a divalent-ion-chelating agent, preferably EDTA.

By way of illustration, the elution buffer may contain a final EDTA concentration of at least 1 mM and of at most 30 mM.

The expression “at least 1 mM” encompasses at least 2, 3, 4, 5, 6, 7, 8, 9 or 10 mM.

The expression “at most 30 mM” encompasses at most 29, 28, 27, 26, 25, 24, 23, 22, 21, 20, 19, 18, 17, 16, 15, 14, 13, 12 or 11 mM.

Furthermore, the production of an affinity substrate comprising a nucleic aptamer of the invention and the implementation of a method for purifying human factor VII with said affinity substrate are illustrated in the examples.

Generally, the solid substrates on which the aptamers of the invention can be immobilized encompass any type of substrate having the structure and the composition commonly found for filter substrates, silicon substrates for chips, membranes, etc. The solid substrates encompass in particular resins, affinity chromatography column resins, polymer beads, magnetic beads, etc. The solid substrates also encompass in particular materials based on glass or metal, such as steel, gold, silver, aluminum, copper, silicon, glass or ceramic. The solid substrates also encompass in particular polymer materials, such as a polyethylene, a polypropylene, a polyamide, a polyvinylidene fluoride, and combinations thereof.

The solid substrate may be coated with a material that facilitates attachment, binding, formation of complexes, immobilization or interaction with the aptamers.

In some embodiments, the solid substrate is a glass slide of which the surface is coated with a layer of gold, with a layer having undergone a treatment by carboxymethylation, with a layer of dextran, of collagen, of avidin, of streptavidin, etc.

In this way, the aptamers according to the invention can be immobilized on the solid substrate by means of an attachment coating, as, for example, described above, either by chemical reaction with the creation of covalent bonds, or by association by means of noncovalent bonds such as hydrogen bonds, electrostatic forces, Van der Waals forces, etc.

According to the invention, the term “affinity substrate” is intended to mean, generally, a substrate made of a solid material on which nucleic aptamers as defined in the present description have been immobilized.

The examples describe embodiments of solid substrates on which the aptamers of the invention are immobilized by means of noncovalent bonds.

The examples describe in particular solid substrates consisting of a glass slide coated with a layer of streptavidin molecules, and aptamers of the invention conjugated to a biotin molecule which are immobilized on the substrate via noncovalent biotin/streptavidin association.

The examples also describe solid substrates consisting of a polystyrene material coated with a layer of streptavidin molecules, and aptamers of the invention conjugated to a biotin molecule which are immobilized on the substrate via noncovalent biotin/streptavidin association.

In some embodiments, the aptamers of the invention can be immobilized on a solid substrate suitable for affinity chromatography, electrochromatography and capillary electrophoresis, as described, for example, by Ravelet et al. (2006, J Chromatogr A, Vol. 117(1): 1-10), Connor et al. (2006, J Chromatogr A, Vol. 111(2): 115-119), Cho et al. (2004, Electrophoresis, Vol. 25 (21-22): 3730-3739) or else Zhao et al. (2008, Anal Chem, Vol. 80(10): 3915-3920).

An aptamer of formula (I) which is at least 15 nucleotides in length and which binds specifically to human factor VII/VIIa, a compound of formula (II), or a compound of formula (III), can also be advantageously used as an agent for capturing human FVII/VIIa, in detection or diagnostic methods and devices.

According to yet another aspect, the present invention also relates to a method for detecting the presence of human factor VII/VIIa in a sample, comprising the following steps:
a) bringing (i) a nucleic acid of formula (I), a compound of formula (II) or a compound of formula (III) or a solid substrate on which a plurality of molecules of said nucleic acid or of said compound are immobilized, into contact with (ii) said sample, and
b) detecting the formation of complexes between (i) said nucleic acid of formula (I), said compound of formula (II) or said compound of formula (III) or said substrate and (ii) the factor VII/VIIa.

The examples of the present patent application provide various embodiments of methods for detecting human FVII/VIIa with aptamers of the invention immobilized beforehand on a solid substrate.

For the implementation of a detection method according to the invention, the solid substrate used may be a solid substrate chosen from the solid substrates described previously in relation to the method for purifying human factor VII/VIIa.

For the implementation of a method or of a device for detecting human factor VII/VIIa, those skilled in the art may refer in particular to the techniques described in European patent application No. EP 1 972 693, PCT application No. WO 2008/038696, PCT application No. WO 2008/025830, or else PCT application No. WO 2007/0322359.

In some embodiments, step b) of detecting the formation of complexes between (i) said nucleic acid or said solid substrate and (ii) human factor VII/VIIa can be carried out by measuring the surface plasmon resonance signal, as is described in the examples.

In some other embodiments, step b) of detecting the formation of complexes between (i) said nucleic acid or said solid substrate and (ii) human factor VII/VIIa can be carried out by bringing said complexes possibly formed into contact with a ligand of human factor VII/VIIa, said ligand being detectable. The examples describe these embodiments in which monoclonal or polyclonal anti-human FVII/VIIa antibodies, labeled with an enzyme, in the case in point horseradish peroxidase, are used as a detectable ligand of human factor VII/VIIa, as is conventionally used in assays of ELISA type.

Advantageously, the sample comprising, or capable of comprising, human factor VII/VIIa consists of a liquid sample which contains the human factor VII/VIIa, including a liquid sample comprising the human factor VII/VIIa and which may also contain molecules of factor VII/VIIa from a nonhuman mammal. In some embodiments of the purification method or the detection method above, said sample consists of a biological solution, such as a body fluid, a cell, a ground cell material, a tissue, a ground tissue material, an organ or a whole organism.

In some embodiments of the purification method or of the detection method above, said sample consists of a liquid biological solution originating from an animal, such as blood, a blood derivative, mammalian milk or a mammalian milk derivative. Said sample can consist of plasma, plasma cryoprecipitate, clarified milk, or derivatives thereof.

In particularly preferred embodiments of the purification method or of the detection method above, said sample originates from an animal that is transgenic for human factor VII/VIIa. Advantageously, the solution is milk from a mammal or a derivative of milk from a mammal that is transgenic for human factor VII/VIIa. For the purpose of the invention, the transgenic animals encompass (i) nonhuman mammals such as cows, goats, rabbits, pigs, monkeys, rats or mice, (ii) birds or else (iii) insects such as mosquitoes, flies or silkworms. In some preferred embodiments, the animal that is transgenic for human factor VII/VIIa is a nonhuman transgenic mammal, entirely preferably a doe rabbit that is transgenic for human factor VII/VIIa. Advantageously, the transgenic mammal produces the recombinant factor VII/VIIa in its mammary glands, owing to the insertion into its genome of an expression cassette comprising a nucleic acid encoding human factor VII/VIIa, which is placed under the control of a specific promoter allowing the expression of the transgenic protein in the milk of said transgenic mammal.

A method for producing human factor VII/VIIa in the milk of a transgenic animal can comprise the following steps: a DNA molecule comprising a gene encoding human factor VII/VIIa, said gene being under the control of a promoter of a protein naturally secreted in the milk (such as the casein promoter, the beta-casein promoter, the lactalbumin promoter, the beta-lactoglobulin promoter or the WAP promoter), is integrated into an embryo of a nonhuman mammal. The embryo is then placed in a mammalian female of the same species. Once the mammal resulting from the embryo is sufficiently developed, lactation by the mammal is induced, and the milk is then collected. The milk then contains the human factor VII/VIIa.

An example of a method for preparing protein in the milk of a mammalian female other than a human being is given in document EP 0 527 063, the teaching of which can be reproduced for producing the protein of the invention. A plasmid containing the WAP (whey acidic protein) promoter is produced by introducing a sequence comprising the promoter of the WAP gene, this plasmid being prepared in such a way as to be able to receive a foreign gene placed under the control of the WAP promoter. The plasmid containing the promoter and the gene encoding the protein of the invention are used to obtain transgenic doe rabbits by microinjection into the male pronucleus of embryos of doe rabbits. The embryos are then transferred into the oviduct of hormonally prepared females. The presence of the transgenes is revealed by the Southern technique using the DNA extracted from the young transgenic rabbits obtained. The concentrations in the milk of the animals are evaluated by means of specific radioimmunological assays.

Other documents describe methods for preparing proteins in the milk of a mammalian female other than a human being. Mention may be made, without being limited thereto, of documents U.S. Pat. No. 7,045,676 (transgenic mouse) and EP 1 739 170 (production of von Willebrand factor in a transgenic mammal).

According to other aspects of the present invention, a nucleic acid which is at least 15 nucleotides in length and which binds specifically to human factor VII/VIIa can be used in vivo, in physiological situations in which it is necessary to bring about an inhibition of the activation of factor X by factor Vila, for example by inhibiting the formation of a functional factor VII/tissue factor complex.

In other words, an aptamer nucleic acid as defined in the present description can also be used, preventively or curatively, as an anticoagulant active ingredient of a medicament.

In particular, an aptamer nucleic acid as defined in the present description can be used as a preventive or curative anticoagulant active ingredient, in particular for the treatment of coagulation disorders including vein thrombosis, arterial thrombosis, post-surgical thrombosis, disorders associated with coronary artery bypass grafts (CABG), strokes, percutaneous transluminal coronary angioplasty (PTCA), tumor metastases, non-severe or severe inflammatory reactions, septic shocks, hypotension, acute respiratory distress syndrome (ARDS), pulmonary embolisms, disseminated intravascular coagulation (DIC), vascular restenoses, platelet deposit, myocardial infarction, angiogenesis, and any prophylactic treatment of men or women who have a risk of developing a thrombosis.

A subject of the present invention is also a pharmaceutical composition comprising a nucleic acid which is at least 15 nucleotides in length and which binds specifically to human factor VII/VIIa as defined in the present description, in combination with one or more pharmaceutically acceptable excipients.

The amount of anti-human FVII/FVIIa aptamer nucleic acid according to the invention, in a pharmaceutical composition, is adjusted so as to allow the administration of an effective amount of this active ingredient to patients.

The ranges of doses of the anti-human FVII/VIIa aptamer of the invention can be readily determined by the physician or the pharmacist. Generally, the amount of active ingredient to be administered varies with the age, the medical condition and the sex of the patient, and also with the extent of the disease and the degree of risk of developing the disease, and can be readily determined by those skilled in the art.

Generally, a pharmaceutical composition comprises an amount of an anti-human FVII/VIIa aptamer ranging from 1 nanogram to 100 milligrams per unit dose, better still ranging from 100 nanograms to 10 milligrams per unit dose.

Generally, a pharmaceutical composition according to the invention comprises from 0.01% to 99.9% by weight of an anti-FVII/VIIa aptamer, or of a combination of several anti-FVII/VIIa aptamers, and from 99.9% to 0.01% by weight of an excipient, or of a combination of excipients, relative to the total weight of said composition.

In some embodiments, said pharmaceutical composition comprises 1, 2, 3, 4, 5 or 6 distinct anti-FVII/VIIa aptamers.

For preparing a pharmaceutical composition according to the invention, those skilled in the art can advantageously refer to the handbook by Remington.

The pharmaceutical composition according to the invention can be used for parenteral, topical or local administration, and prophylactically and/or therapeutically. Thus, an anti-human FVII/VIIa aptamer according to the present invention is prepared in a form suitable for the type of administration selected, for example in liquid form or in freeze-dried form. The pharmaceutical compositions of anti-human FVII/VIIa aptamers according to the present invention can contain an excipient and/or a vehicle which is pharmaceutically acceptable, preferably aqueous. Many excipients and/or vehicles which are pharmaceutically acceptable can be used, for example water, buffered water, a saline solution, a glycine solution, and derivatives thereof, and also agents necessary for reproducing the physiological conditions, for instance buffering agents and pH adjusters, surfactants such as sodium acetate, sodium lactate, sodium chloride, potassium chloride or calcium chloride, this list not being limiting. Furthermore, the pharmaceutical composition can be sterilized by sterilization techniques well known to those skilled in the art. Generally, in order to produce a pharmaceutical composition in accordance with the invention, those skilled in the art can advantageously also refer to the latest edition of the European Pharmacopeia, for example to the 5th edition of the European Pharmacopeia published in January 2005, or else to the 6th edition of the European Pharmacopeia, available to the public in June 2007.

For the production of a pharmaceutical composition comprising an aptamer as active ingredient, those skilled in the art can also refer to the content of PCT application No. WO 2007/058801, PCT application No. WO 2005/084412 and PCT application No. WO 2004/047742, or else in PCT application No. WO 2008/150495.

In some embodiments of a pharmaceutical composition according to the invention, the anti-human FVII/VIIa aptamer active ingredient(s) may be used alone or in combination with one or more other pharmaceutically active molecules, including with one or more other anticoagulant active ingredients.

The invention also relates to a nucleic acid which is at least 15 nucleotides in length and which binds specifically to human factor VII/VIIa, as defined in the present description, for its use as a medicament.

The invention also relates to the use of a nucleic acid which is at least 15 nucleotides in length and which binds specifically to human factor VII/VIIa, as defined in the present description, for producing a medicament for the treatment of coagulation disorders.

The invention also relates to the use of a nucleic acid which is at least 15 nucleotides in length and which binds specifically to human factor VII/VIIa, as defined in the present description, for the treatment of coagulation disorders.

A subject of the invention is also a method for preventing or treating a coagulation disorder, comprising a step during which an anti-human FVII/VIIa aptamer pharmaceutical composition as defined in the present description is administered to a patient requiring a preventive or curative treatment for a coagulation disorder.

Generally, a daily dose of an anti-human FVII/VIIa aptamer as defined in the present description ranging from 1 nanogram to 100 milligrams, better still ranging from 100 nanograms to 10 milligrams, is administered for a patient weighing 80 kg.

The present invention is also illustrated hereinafter in the following examples, without being limited thereto.

### EXAMPLES

### Example 1

### Capture of Aptamer Nucleic Acids According to the Invention by Human Factor VII/VIIa Immobilized on a Substrate

A solid substrate on which molecules of purified human factor VII/VIIa of plasma origin were immobilized was produced.

The human factor VII is immobilized on carboxymethyl dextran activated with NHS-EDC and which binds to the free amines present on the FVII/VIIa.

The human factor VII/VIIa is thus immobilized with a degree of immobilization of 2743 RU (1 RU corresponds approximately to 1 pg of product immobilized per mm²).

Nucleic aptamers of the invention (purity: 99%), respectively the Mapt2 aptamer (SEQ ID No. 86), the Mapt3 aptamer (SEQ ID No. 41) and the Mapt7 aptamer (SEQ ID No. 58), were diluted in running buffer (50 mM Tris, 50 mM NaCl, 10 mM CaCl₂, 4 mM MgCl₂, pH 7.4) so as to obtain three aptamer samples.

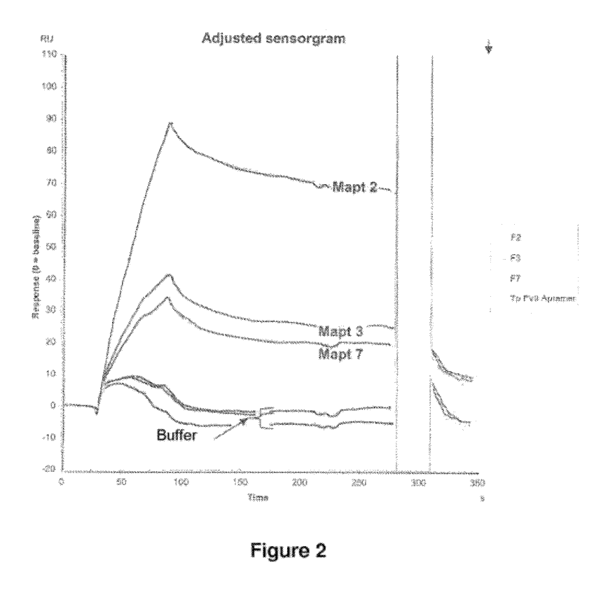

Each sample was injected sequentially onto the same chip (solid substrate) containing the immobilized human FVII. Controls were obtained by injecting blanks containing only running buffer. All the injections were carried out with a flow rate of 30 μl/min for 60 sec; after the injection, running buffer was injected onto the chip at an identical flow rate, for 120 sec. Elution buffer (5 mM EDTA) was then injected for 60 sec with a flow rate of 30 μl/min in order to detach the immobilized human FVII aptamer. The chip makes it possible to study, in real time, the formation and the breaking of the interactions between the immobilized human FVII and each of the aptamers Mapt2, Mapt3 and Mapt7 tested, by means of surface plasmon resonance (SPR). Binding to the immobilized human FVII is reflected by an increase in the signal, in resonance units (RU), recorded by the apparatus (FIG. 2). These analyses are carried out with the RPS Biacore T100 apparatus (GE). The modeling of the interactions recorded is carried out using the Biaevaluation software (GE).

The results obtained show that all the nucleic aptamers tested bind with significant affinity to the human plasma factor VII.

### Example 2

### Capture of Human Factor VII/VIIa by an Aptamer Nucleic Acid According to the Invention, Immobilized on a Substrate

A solid substrate on which molecules of the nucleic aptamer of the invention of sequence SEQ ID No. 86, also denoted herein “Mapt2”, were immobilized, was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (Series S sensor Chip SA, GE).

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 983 RU (1 RU corresponds approximately to 1 pg of product immobilized per mm₂).

Human FVII purified from plasma (FVII HP, purity: 99%) was diluted in running buffer (50 mM Tris, 50 mM NaCl, 10 mM CaCl₂, 4 mM MgCl₂, pH 7.4) so as to obtain four samples having an FVII HP concentration of 62, 125, 250 and 500 nM.

Separately, a preparation of polyvalent immunoglobulins (Tegeline®, sold by LFB, France) was diluted in running buffer (50 mM Tris, 50 mM NaCl, 10 mM CaCl₂, 4 mM MgCl₂, pH 7.4) so as to obtain four samples having a concentration of polyvalent immunoglobulins of 62, 125, 250 and 500 nM.

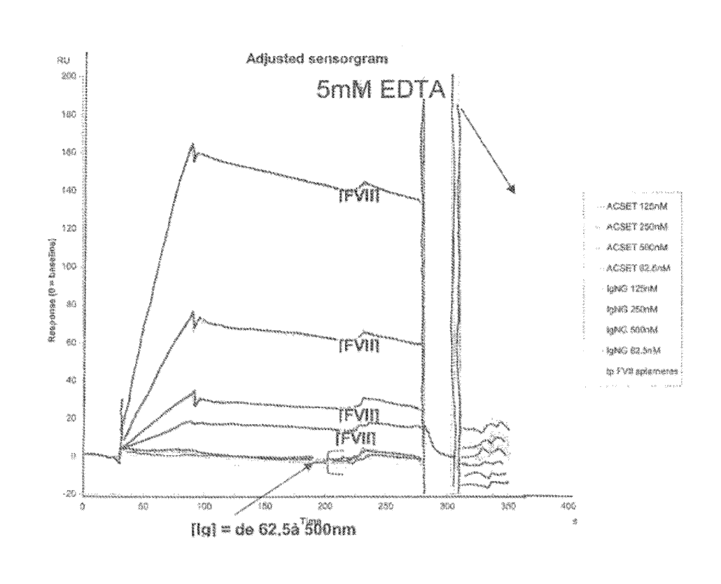

Each sample was injected sequentially onto the same chip (solid substrate) containing the Mapt2 aptamer immobilized by means of a biotin-streptavidin interaction. Controls were obtained by injecting blanks containing only running buffer. All the injections were carried out with a flow rate of 30 μl/min for 60 sec; after the injection, running buffer was injected onto the chip at an identical flow rate, for 120 sec. Elution buffer (5 mM EDTA) was then injected for 30 sec with a flow rate of 30 μl/min in order to detach the FVII HP from the aptamer. The chip makes it possible to study, in real time, the formation and the breaking of the interactions between the FVII HP, or the polyvalent immunoglobulins, and the immobilized aptamer, by means of surface plasmon resonance (SPR). Binding to the immobilized aptamer is reflected by an increase in the signal, in resonance units (RU), recorded by the apparatus (FIG. 3). These analyses are carried out with the RPS Biacore T100 apparatus (GE). The modeling of the interactions recorded is carried out using the Biaevaluation software (GE).

This example shows that the Mapt2 aptamer, once immobilized, binds specifically to the FVII HP, with a significant affinity. The Mapt2 aptamer does not bind to the polyvalent immunoglobulins.

### Example 3

### Capture of Human Factor VII/VIIa by an Aptamer Nucleic Acid According to the Invention, Immobilized on a Substrate

A solid substrate on which molecules of the nucleic aptamer of the invention of the sequence SEQ ID No. 86, also denoted herein “Mapt2”, were immobilized, was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (Series S sensor Chip SA, GE).

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 424.9 RU (1 RU corresponds approximately to 1 pg of product immobilized per mm²).

Human FVII purified from plasma (FVII HP, purity: 99%) was diluted in running buffer (50 mM Tris, 50 mM NaCl, 10 mM CaCl₂, 4 mM MgCl₂, pH 7.4) so as to obtain a sample having an FVII HP concentration of 500 nM.

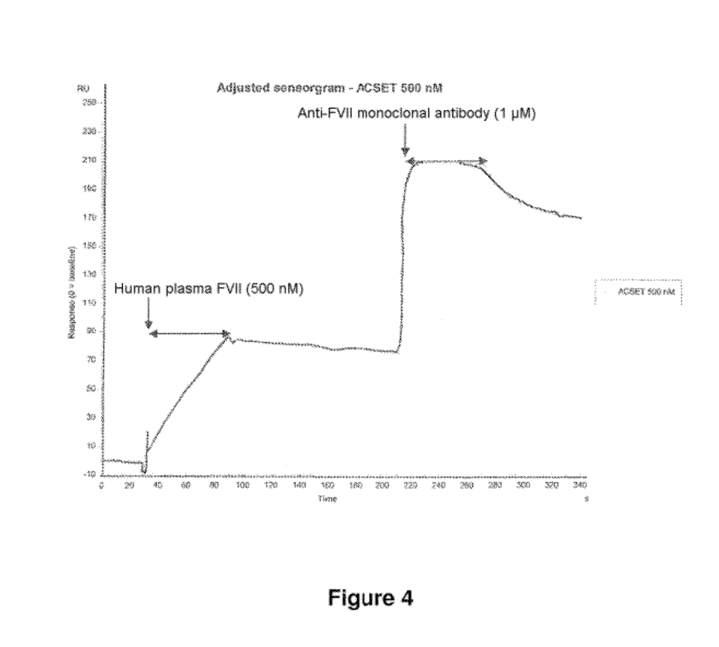

Each sample was injected sequentially onto the same chip (solid substrate) containing the Mapt2 aptamer immobilized by means of a biotin-streptavidin interaction. Controls were obtained by injecting blanks containing only running buffer. All the injections were carried out with a flow rate of 30 μl/min for 60 sec; after the injection, running buffer was injected onto the chip at an identical flow rate, for 120 sec. Elution buffer (5 mM EDTA) was then injected for 30 sec with a flow rate of 30 μl/min in order to detach the FVII HP from the aptamer. The chip makes it possible to study, in real time, the formation and the breaking of the interactions between the FVII HP and the immobilized aptamer, by means of surface plasmon resonance (SPR). Binding to the immobilized aptamer is reflected by an increase in the signal, in resonance units (RU), recorded by the apparatus (FIG. 4). These analyses are carried out with the RPS Biacore T100 apparatus (GE).

In order to verify that the product which binds to the immobilized aptamer is indeed the FVII, an injection sequence comprising (i) the FVII HP at 500 nM and (ii) an anti-FVII monoclonal antibody at 1 μM (Sigma, Ref Clone No. MC1476/E.A.8.1) was carried out on the same chip. The injection and analysis conditions are identical to those previously described. If the aptamer actually retains FVII, the injection of anti-FVII monoclonal antibody should be reflected by an increase in the signal in RU as a result of the binding of the antibodies to the FVII, itself bound to the aptamer. Antibodies alone are injected as controls. The increase in signal in RU clearly shows that the aptamer recognizes the FVII (FIG. 4).

The modeling of the interactions recorded is carried out using the Biaevaluation software (GE).

The results of this example show that the aptamer, once immobilized, binds specifically to the FVII HP, with a significant affinity, and that the signal observed is indeed due to the retention of the FVII on the aptamer.

### Example 4

### Mapt2 Aptamer/Human Plasma FVII Stoichiometry

A solid substrate on which molecules of the nucleic aptamer of the invention of sequence SEQ ID No. 86, also denoted herein “Mapt2”, were immobilized, was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (Series S sensor Chip SA, GE).

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 983 RU (1 RU corresponds approximately to 1 pg of product immobilized per mm²).

Human FVII purified from plasma (FVII HP, purity: 99%) was diluted in running buffer (50 mM Tris, 50 mM NaCl, 10 mM CaCl₂, 4 mM MgCl₂, pH 7.4) so as to obtain a sample which has an FVII HP concentration of 1 μM.

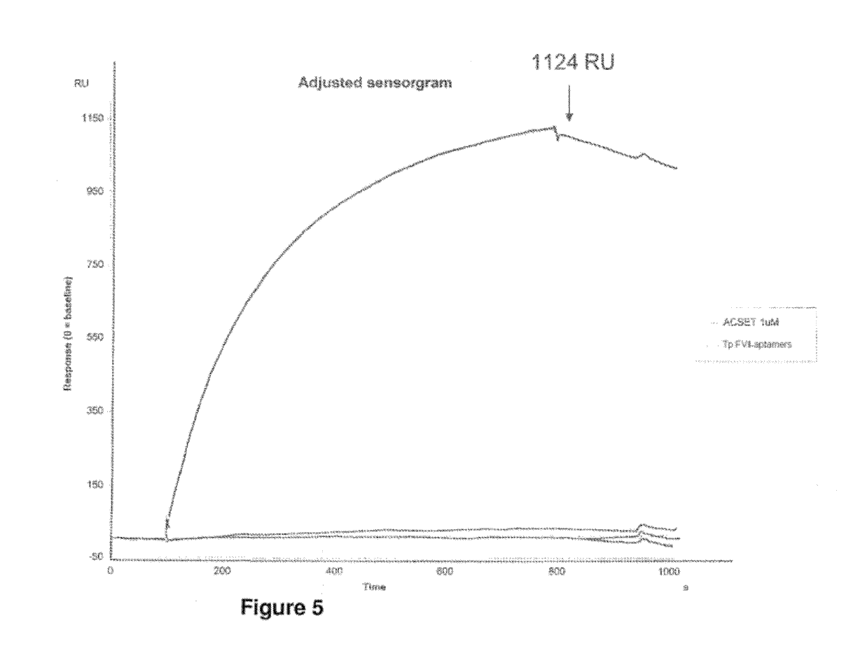

Each sample was injected sequentially onto the same chip (solid substrate) containing the Mapt2 aptamer immobilized by means of a biotin-streptavidin interaction. Controls are obtained by injecting blanks containing only running buffer. All the injections were carried out with a flow rate of 30 μl/min for 700 sec; after the injection, running buffer was injected onto the chip at an identical flow rate, for 120 sec. Elution buffer (20 mM EDTA) was then injected for 30 sec with a flow rate of 30 μl/min in order to detach the FVII HP from the aptamer. The chip makes it possible to study, in real time, the formation and the breaking of the interactions between the FVII HP and the immobilized aptamer, by means of surface plasmon resonance (SPR). Binding to the immobilized aptamer is reflected by an increase in the signal, in resonance units (RU), recorded by the apparatus (FIG. 5). These analyses are carried out with the RPS Biacore T100 apparatus (GE).

The modeling of the interactions recorded is carried out using the Biaevaluation software (GE).

The results of this example show that increasing amounts of human FVII bind to the Mapt2 aptamer over the course of the injection time and that saturation of the aptamer sites by the human FVII molecules is not yet reached at the end of the injection period (700 sec).

The results show that a large part of the Mapt2 aptamer molecules immobilized at the surface of the solid substrate are capable of binding human FVII. These results signify that the conformation in which the aptamer is capable of binding its target is sufficiently stable for the vast majority of the aptamers to be in this form, and that this conformation is not significantly detrimentally modified or impaired by the immobilization.

These results also show that the Mapt2/human FVII binding stoichiometry is approximately 1/1.

For the Mapt2/human FVII binding, the maximum signal achieved was:

[MW FVII/MW Mapt2]*level of immobilization*stoichio, with:

MW FVII signifies the molecular weight of the human FVII, equal to 50 kDa

MW Mapt2 signifies the molecular weight of Mapt2, equal to 27 kDa,

level of immobilization is, in this example, 983.3, and

stoichio signifies the Mapt2/FVII stoichiometry, which is equal to 1.

With the formula above, the value of the maximum signal achieved was: 1820 RU.

The percentage of Mapt2 aptamers having bound a molecule of human factor VII is then calculated according to the formula: measured signal/expected signal, with:

measured signal which is equal to 1124 RU, and

expected signal which is equal to 1820 RU

According to the formula above, the percentage of molecules of the Mapt2 aptamer having bound a molecule of human FVII at the end of the injection is 62%.

### Example 5

### Kinetics of Binding of the Mapt2 Aptamer to Human Plasma FVII

A solid substrate on which molecules of the nucleic aptamer of the invention of sequence SEQ ID No. 86, also denoted herein “Mapt2”, were immobilized, was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (Series S sensor Chip SA, GE).

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 425 RU (1 RU corresponds approximately to 1 pg of product immobilized per mm²).

Human FVII purified from plasma (FVII HP, purity: 99%) was diluted in running buffer (50 mM Tris, 50 mM NaCl, 10 mM CaCl₂, 4 mM MgCl₂, pH 7.4) so as to obtain four samples having an FVII HP concentration of 125, 250 (in duplicate) and 500 nM and 1000 mM.

Each sample was injected sequentially onto the same chip (solid substrate) containing the Mapt2 aptamer immobilized by means of a biotin-streptavidin interaction. Controls are obtained by injecting blanks containing only running buffer. All the injections were carried out with a flow rate of 30 μl/min for 60 sec; after the injection, running buffer was injected onto the chip at an identical flow rate, for 120 sec. Elution buffer (20 mM EDTA) was then injected for 75 sec with a flow rate of 30 μl/min in order to detach the FVII HP from the aptamer.

These analyses are carried out with the RPS Biacore T100 apparatus (GE). The modeling of the interactions recorded is carried out using the Biaevaluation software (GE).

The curves of the kinetics of binding of the immobilized Mapt2 aptamer to the human plasma FVII were calculated with the dedicated module of the Biacore® control Software, version 1.2.

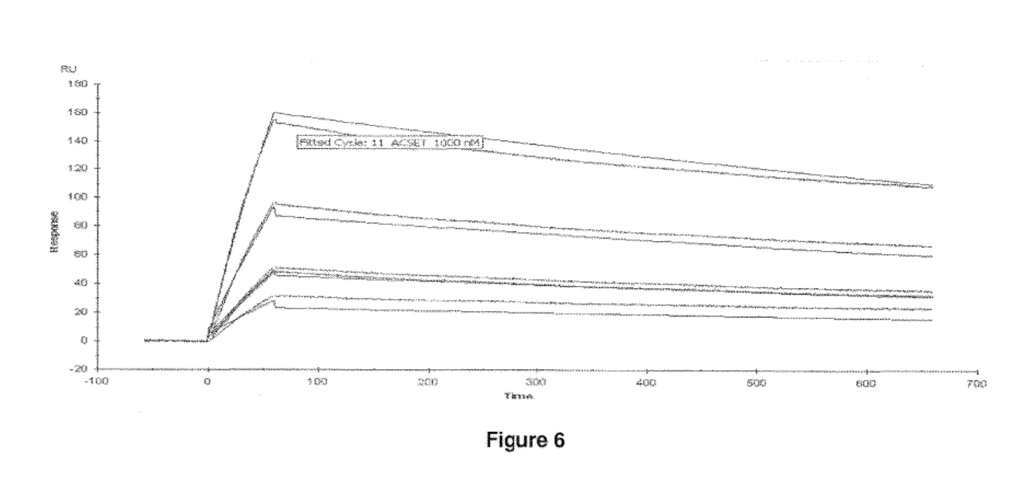

The curves of the kinetics of binding of the immobilized Mapt2 aptamer to the human plasma FVII are represented in FIG. 6.

The results of the calculation of the kinetics of binding of the Mapt2 aptamer to the human FVII made it possible to determine that:

the dissociation constant Kd of Mapt2 is 99.9 nM, and

the association constant Ka (=6.25×10³ M⁻¹s⁻¹) of Mapt2 is 6.25×10⁻⁴s⁻¹

### Example 6

### Elution of Recombinant Human FVII with EDTA

A solid substrate on which molecules of the nucleic aptamer of the invention of sequence SEQ ID No. 86, also denoted herein “Mapt2”, were immobilized, was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided.

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

A solid substrate on which molecules of a biotinylated anti-FVII polyclonal antibody were immobilized, was also produced.

The solid substrates with the Mapt2 aptamer or with the anti-human FVII polyclonal antibody consist of 96-well plates for ELISA assay.

An anti-human FVII polyclonal antibody labeled with horseradish peroxidase was used for the visualization.

The assay conditions are detailed below.

plate: Biobind Streptavidin coated (Thermo ref: 95029293) buffers:

immobilization: 50 mM Tris, 150 mM NaCl, 0.1% Tween 20/pH=7.5 Ca²⁺/Mg²⁺ washing: 50 mM Tris, 50 mM NaCl, 10 mM CaCl₂, 4 mM MgCl₂, 0.1% Tween 20/pH=7.4

EDTA washing: 10 mM EDTA in injectable water+0.1% Tween 20

ligand: Mapt2 obtained by chemical synthesis at Eurogentec. Concentration for immobilization 200 nM, volume=100 μl, 1 h at ambient temperature

control ligands: anti-FVII polyclonal antibody purified on FVII affinity chromatography (R&D systems, ref: BAF 2338). Concentration for immobilization 200 nM, volume=100 μl, 1 h at ambient temperature

sample: transgenic human FVII produced in rabbits, stabilized with 1% BSA, batch: 479186. Concentration=100 nM, volume deposited=100 μl, 1H 15 at ambient temperature

visualizing antibody: derived from the Asserachrom FVII:Ag kit (diagnostica Stago) prepared according to the supplier's recommendations. Volume=100 μl, 45 min at ambient temperature

visualization: 100 μl per well of a solution of OPD+H₂O₂

reaction stop: H₂SO₄, 50 μl per well

reading at 492 nm.

The results, expressed as OD at 492 nm, are given in table 1 below.

TABLE 1

| Washing: Ca²⁺/Mg²⁺ | Washing: EDTA |
|---|---|
| 3.53/3.56 | 0.093/0.09 |
| 3.53/3.79 | 0.094/0.085 |
| 3.75/3.75 | 0.097/0.09 |
| 3.9/4.0 | 3.45/3.27 |
| 4.0/4.2 | 3.52/3.54 |

results in bold characters: immobilized Mapt2 aptamer (SEQ ID No. 86)

results in normal characters: immobilized anti-human FVII polyclonal antibody

The results of this example show that only binding of the human FVII with the Mapt2 aptamer allows elution with EDTA.

### Example 7

### Binding of the Aptamers to Various Types of Human Factor VII

A solid substrate on which molecules of the nucleic aptamer of the invention of sequence SEQ ID No. 86, also denoted herein “Mapt2”, were immobilized, was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (Series S sensor Chip SA, GE).

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 4326 RU (1 RU corresponds approximately to 1 pg of product immobilized per mm²). The part of the solid substrate on which Mapt2 is immobilized is called the active cell.

According to the same technique, some nucleic acid molecules or other were immobilized, with a degree of immobilization of 4069 RU, on an independent part of the solid substrate, called the reference cell.

Various types of human factor VII were used, respectively:

human plasma FVII obtained according to the Acset purification method,

recombinant human FVII produced in rabbits,

recombinant human FVII produced in goats, and

recombinant human FVII (Novoseven® sold by Novo).

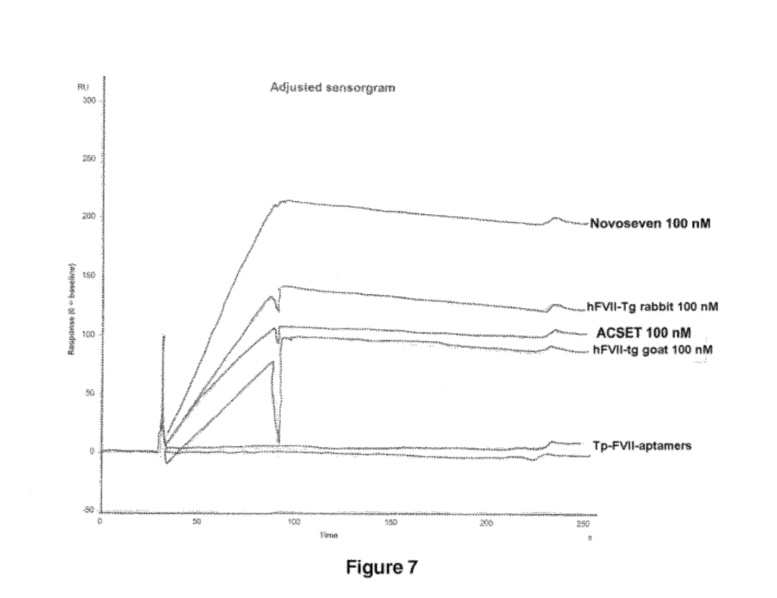

Each sample was injected sequentially onto the same active cell (solid substrate) containing the Mapt2 aptamer immobilized by means of a biotin-streptavidin interaction. Controls are obtained by injecting blanks containing only running buffer. Signals corresponding to the background noise are obtained by injecting the same samples onto the reference cell containing the immobilized some nucleic acids or other, these signals are subtracted from the signals obtained on the active cell. All the injections were carried out with a flow rate of 30 μl/min for 60 sec; after the injection, running buffer was injected onto the chip at an identical flow rate, for 120 sec. Elution buffer (15 mM EDTA) was then injected for 30 sec with a flow rate of 30 μl/min in order to detach the FVII from the aptamer. The chip makes it possible to study, in real time, the formation and the breaking of the interactions between the FVII and the immobilized aptamer by means of surface plasmon resonance (SPR). Binding to the immobilized aptamer is reflected by an increase in the signal, in resonance units (RU), recorded by the apparatus (FIG. 7). These analyses are carried out with the RPS Biacore T100 apparatus (GE).

The modeling of the interactions recorded is carried out using the Biaevaluation software (GE).

The results are represented in FIG. 7.

The results of this example show that the aptamer, once immobilized, binds specifically to a large variety of human factor VII, including human plasma factor VII and recombinant human factors VII produced in various transgenic animals, including rabbits and goats.

### Example 8

### Specific Binding of the Aptamers to Human Factor VII

A solid substrate on which molecules of the nucleic aptamer of the invention of sequence SEQ ID No. 86, also denoted herein “Mapt2”, were immobilized, was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (Series S sensor Chip SA, GE).

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 4326 RU (1 RU corresponds approximately to 1 pg of product immobilized per mm²).

The part of the solid substrate on which Mapt2 is immobilized is called the active cell.

According to the same technique, some nucleic acid molecules or other were immobilized, with a degree of immobilization of 4069 RU, on an independent part of the solid substrate, called the reference cell.

Various types of factor VII were used, respectively:
human plasma FVII obtained according to the Acset purification method,
recombinant rabbit FVII sold by the company American Diagnostica (ref 407RAB, batch No. 080818).

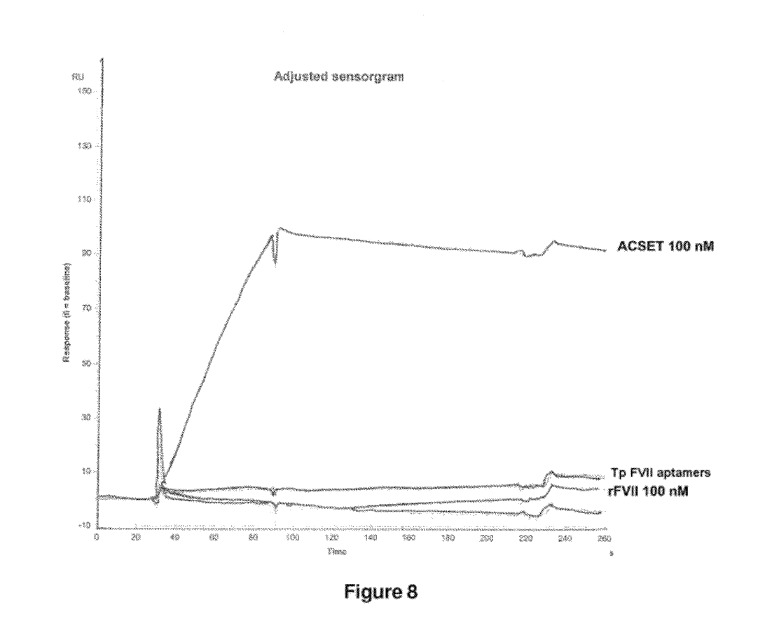

Each sample was injected sequentially onto the same chip (solid substrate) containing the Mapt2 aptamer immobilized by means of a biotin-streptavidin interaction. Controls are obtained by injecting blanks containing only running buffer. Signals corresponding to the background noise are obtained by injecting the same samples onto the reference cell containing the immobilized some nucleic acids or other, these signals are subtracted from the signals obtained on the active cell. All the injections were carried out with a flow rate of 30 μl/min for 60 sec; after the injection, running buffer was injected onto the chip at an identical flow rate, for 120 sec. Elution buffer (15 mM EDTA) was then injected for 30 sec with a flow rate of 30 μl/min in order to detach the human or rabbit FVII from the aptamer. The chip makes it possible to study, in real time, the formation and the breaking of the interactions between the FVII HP and the immobilized aptamer by means of surface plasmon resonance (SPR). Binding to the immobilized aptamer is reflected by an increase in the signal, in resonance units (RU), recorded by the apparatus (FIG. 8). These analyses are carried out with the RPS Biacore T100 apparatus (GE).

The modeling of the interactions recorded is carried out using the Biaevaluation software (GE).

The results of this example show that the aptamer, once immobilized, is highly specific for human FVII/VIIa and does not bind to rabbit FVII/VIIa.

### Example 9

### Preparation of an Affinity Substrate

The affinity substrate was prepared from a solid substrate material consisting of a matrix onto which streptavidin (streptavidin-agarose—Novagen®) was grafted.

A volume of 1 ml of gel was placed in a container consisting of a column (i.d. 11 mm). The gel was washed with purified water, in order to remove the storage solvent.

The characteristics of the gel are:
biotin adsorption capacity: ≧85 nanomol/ml of gel
functional test: capture>99% of biotinylated thrombin over the course of 30 minutes at AT
other tests: protease-free, endo/exonuclease-free, RNase-free
preservative: 100 mM sodium phosphate pH 7.5+NaN₃ 0.02

The outlet of the packed column (gel bed height=1 cm) is connected to an absorbance detector equipped with a UV filter at 254 nm and a recording device.

The biotinylated anti-human FVII nucleic aptamers of sequence SEQ ID No. 86 are solubilized in purified water at a final concentration of 0.5 mg/0.187 ml, i.e. a final molar concentration of 0.1 mM. The solution of nucleic aptamers was activated at 95° C. according to the standard cycle, for the immobilization of the aptamers on the solid substrate material.

The solution of nucleic aptamers was prediluted with 4.8 ml of purified water and then 1.5 ml of Me⁺⁺ buffer (5× concentrated).

The absorbance detector is adjusted to 1 AUFS (absorbance unit full scale) and the OD at 254 nm of this solution is recorded at 0.575 AU₂₅₄.

The solution of biotinylated nucleic aptamers is injected onto the prepacked streptavidin-agarose gel and recirculated with a peristaltic pump at a flow rate of 2.5 ml/minute, i.e. a contact time on the gel of 24 seconds (inlet/outlet I/O). Under these conditions, the OD at 254 nm stabilizes rapidly at 0.05 AU₂₅₄, i.e. a theoretical coupling value of 91%, i.e. 0.455 mg of nucleic aptamers per milliliter of gel.

Washing with a 10 mM CaCl₂+4 mM MgCl₂ buffer and then in 2M NaCl is carried out in order to eliminate the nucleic aptamers which are not bound specifically to the streptavidin molecules grafted onto the solid substrate material.

### Example 10

### Method for Purifying Recombinant Human Factor VII

The aptamer affinity substrates were tested using a purified preparation of FVII/FVIIa prepared according to the technique described in PCT application No. WO2008/099077.

### Preparation of the Sample to be Purified

The starting biological material is transgenic rabbit milk containing recombinant human FVII. The expression cassette comprises the human FVII transgene placed under the control of the 6-casein gene promoter.

Briefly, 140 milliliters of milk were collected from 2 rabbits in first lactation between day 4 and day 12 after having given birth.

The average titer of amidolytic FVII (biologically activatable FVII) in the milk collected is 928 IU/ml. The milks are stored at a temperature of −80° C.

For the test, the rabbit milks are thawed in a water bath at a temperature of 37° C., and are then diluted with a sodium citrate solution to give a final citrate concentration of 62 g/l at a pH of 7.5.

The treatment with sodium citrate makes it possible to destabilize the phosphocalcic casein micelles.

The lipid-rich protein solution of milk is then clarified over a sequence of filters, respectively (i) depth filter of 15 to 0.5 μm porosity threshold and then (i) membrane filter at 0.2 μm.

A volume of 360 ml of filtered solution having an FVII titer of 198 IU/ml, i.e. 36 mg of transgenic FVII, is prepurified on an MEP-HyperCel® chromatography gel (Pall BioSepra) having a volume of 16 ml. This capture gel makes it possible to eliminate 95% of the milk proteins, including the majority of caseins, while at the same time retaining 60% of the initial amount of FVII.

An amount of 17.5 mg of low-purity FVII (˜5%) obtained at the end of the above step is purified by ion exchange chromatography, using a Q-sepharose® XL gel (GE Healthcare) having a volume of 20 ml, the human FVII being eluted with a volume of 78 ml of a buffer comprising 5 mM of calcium chloride. The concentration of amidolytic FVII is 337 IU/ml, i.e. 0.17 mg of FVII/ml, and the concentration of total proteins is estimated at 0.18 mg/ml by measurement of OD at 280 nm and ε^{1%}=13, i.e. an FVII purity of 94%.

The residual proteins originating from the rabbit milk are difficult to separate from the FVII at this stage, either because there are structural homologies, such as GLA-domain or EGF-domain proteins, or else because there are physicochemical homologies (similar ionic charge and/or molecular size). Conventional techniques allow an improvement in the purity up to 99.95% by means of orthogonal techniques (combination of separation on hydroxyapatite gel and by size exclusion chromatography). However, for repeated injection in humans, the load with respect to exogenous proteins accepted for genetic recombination proteins must not exceed 50 ppm, i.e. a purity >99.995%. Such a purity appears to be attainable only after purification on an affinity matrix.

### Step of Purifying Recombinant Human FVII on the Affinity Substrate of the Invention

A volume of 6 ml of the solution of purified human FVII (1.1 mg of FVII) obtained at the end of the preceding step is used for the step of purifying the recombinant human FVII at a high level of purity with the affinity substrate of the invention.

The FVII solution obtained in the preceding step, preadjusted to 4 mM MgCl₂ and 10 mM CaCl₂ and pH 7.5, is injected onto the aptamer-agarose gel (affinity substrate) with a peristaltic pump at a flow rate of 0.1 ml/minute, i.e. a contact time with the affinity substrate of 10 minutes (I/O).

After injection, the gel is washed in 50 mM tris+50 mM NaCl+4 mM MgCl₂+10 mM CaCl₂ buffer at pH 7.5.

A nonadsorbed volume of 10 ml of solution is collected.

The FVII is eluted with a 50 mM tris+10 mM EDTA buffer at pH 7.5. The collection of the elution peak is carried out according to the OD profile.

According to the molar calculations, the amount of nucleic aptamers immobilized in the affinity substrate is 17 nanomol, which corresponds, for a mole-for-mole interaction with the FVII molecules, to an absolute capacity of the affinity substrate of 0.9 mg of FVII.

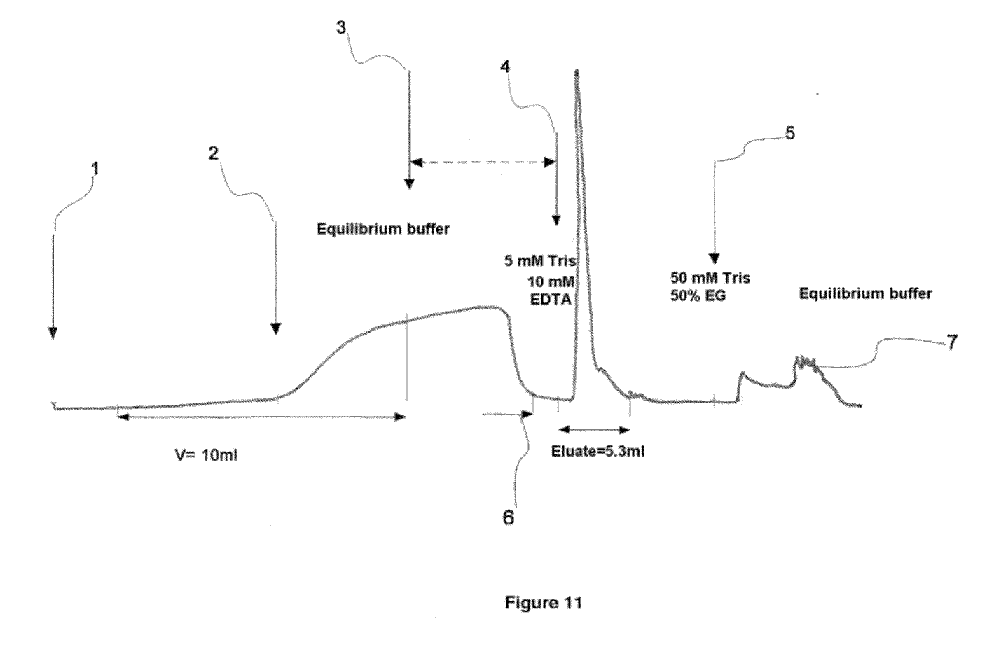

FIG. 11 illustrates a chromatography profile of the recombinant human FVII produced in the rabbit milk, with continuous monitoring of the absorbance values (OD) at 254 nanometers.

In FIG. 11, the inflection (2) of the absorption curve, after the moment of the injection (1), illustrates the beginning of the saturation of the affinity substrate with the recombinant human FVII. At time (3), the injection of recombinant human FVII is stopped. To illustrate the linear scale of the times in FIG. 1, it is indicated that the duration between the injection start time (1) and the injection end time (2) is 10 minutes. The affinity substrate continues to be saturated with the coagulation protein of interest: complexes between (i) the anti-FVII nucleic aptamers of the affinity substrate and (ii) the molecules of recombinant human FVII initially contained in the composition to be purified have been formed. After the composition to be purified has been passed over the column, a step of washing (6) the column with the washing buffer specified above is carried out. The elution step is then carried out, by injection, at time (4), of the buffer solution comprising a final EDTA concentration of 10 mM. The absorption peak illustrates the release of the recombinant human FVII from the nucleic aptamer/recombinant FVII complexes. It is noted that the molecules of recombinant human FVII are released rapidly and therefore in a small volume. Consequently, by virtue of the affinity substrate of the invention, an elution solution with a high concentration of recombinant human FVII protein is obtained. At time (5), a step of regenerating the affinity substrate is carried out with a 50 mM Tris buffer. The absorbance peak visible at (7) corresponds to the substances released from the affinity substrate owing to the regeneration step.

### Dynamic Binding Capacity of the Affinity Substrate

Table 2 below gives the results of the test, which show a dynamic binding capacity of 0.45 to 0.49 mg/ml of the affinity matrices, i.e. 50 to 55% of bioavailable ligands.

In EDTA, a dynamic elution of approximately 75% is calculated.

TABLE 2
Recombinant human FVII and total protein results of the
aptamer-agarose matrix tests:

| | Recombinant | | Proteins | | DBC | |
|---|---|---|---|---|---|---|
| | FVII | | (total mg) | | (mg/ml) | DE (%) |
| Start | 2228 | 100% | 1.42 | 100% | | |
| Final | | | | | | |
| Nonadsorbed | 924 | 41% | 0.57 | 40% | | |
| Eluate | 971 | 44% | 0.61 | 43% | 0.49 | 74% |
| Results by | | 85% | | 83% | | |
| weight | | | | | | |

start: starting sample
final: fraction composition
DBC: dynamic binding capacity
DE: dynamic elution; which represents the ratio between the eluted recombinant FVII and the adsorbed recombinant FVII, expressed as a percentage

### Specific Separation Capacity of the Affinity Substrate

The affinity substrates were evaluated in terms of specificity by means of an ELISA assay specific for rabbit milk proteins.

The results are represented in table 3 below.

TABLE 3
Affinity substrate specificity results:

| | Recombinant FVII | | Rabbit milk | | RMP | % FVII |
|---|---|---|---|---|---|---|
| | (total mg) | | proteins (RMP) | | (ppm) | purity |
| Start | 1.11 | 100% | 16992 | 100% | 16782 | 98.32% |
| Final | | | | | | |
| Nonadsorbed | 0.46 | 41% | 14590 | 40% | 34738 | 96.53% |
| Eluate | 0.49 | 44% | 217 | 43% | 492 | 99.95% |
| Results by | | 85% | | 83% | | |
| weight | | | | | | |

The results in table 3 above show that an average of 2 log₁₀ of elimination by the aptamer-agarose is obtained, taking the purity of the transgenic human FVII from 98.3% to 99.95%. This shows a good specificity of the aptamers with respect to human FVII and very few interactions with the residual rabbit milk proteins.

An improvement is possible by means of intermediate washes, before elution, with solutions such as 2M NaCl and/or propylene glycol or ethylene glycol at 50% if, under these conditions, the FVII is not eluted.

The results of example 10 illustrate the excellent characteristics of the affinity substrates on aptamer-agarose gel with a dynamic binding capacity of at least 1 mg of FVII per mg of ligand with an elution yield of at least 75%. The specificity is also well established with a clear improvement in purity (˜99.95%), with an elimination of 2 log₁₀ of the residual rabbit milk proteins RMP. The final level comes to approximately 500 ppm over these 2 nonoptimized tests.

### Example 11

### Capture of Aptamer Nucleic Acids According to the Invention By Human Factor VII/VIIA Immobilized on a Substrate

A solid substrate on which molecules of purified human factor VII/VIIa of plasma origin were immobilized, was produced.

The human factor VII is immobilized on carboxymethyl dextran activated with NHS-EDC and which binds to the free amines present on the FVII/VIIa.

The human factor VII/VIIa is thus immobilized with a degree of immobilization of 2525 RU (1 RU corresponds approximately to 1 pg of product immobilized per mm²).

A series of 27 nucleic aptamers of the invention (purity: 99%), having a length ranging from 43 to 66 nucleotides depending on the aptamers, were diluted in running buffer (50 mM Tris, 50 mM NaCl, 10 mM CaCl₂, 4 mM MgCl₂, pH 7.5) so as to obtain three aptamer samples. Each aptamer tested was injected at a final concentration of 1 μM in the running buffer.

Each sample was injected sequentially onto the same chip (solid substrate) containing the immobilized human FVII. Controls are obtained by injecting blanks containing only running buffer. All the injections were carried out with a flow rate of 30 μl/min for 60 sec; after the injection, running buffer was injected onto the chip at an identical flow rate, for 120 sec. Elution buffer (5 mM EDTA) was then injected for 60 sec with a flow rate of 30 μl/min in order to detach the aptamer from the immobilized human FVII. The chip makes it possible to study, in real time, the formation and the breaking of the interactions between the immobilized human FVII and each of the aptamers tested, by means of surface plasmon resonance (SPR). Binding to the immobilized human FVII is reflected by an increase in the signal, in resonance units (RU), recorded by the apparatus (figure E1). These analyses are carried out with the RPS Biacore T100 apparatus (GE). The modeling of the interactions recorded is carried out using the Biaevaluation software (GE).

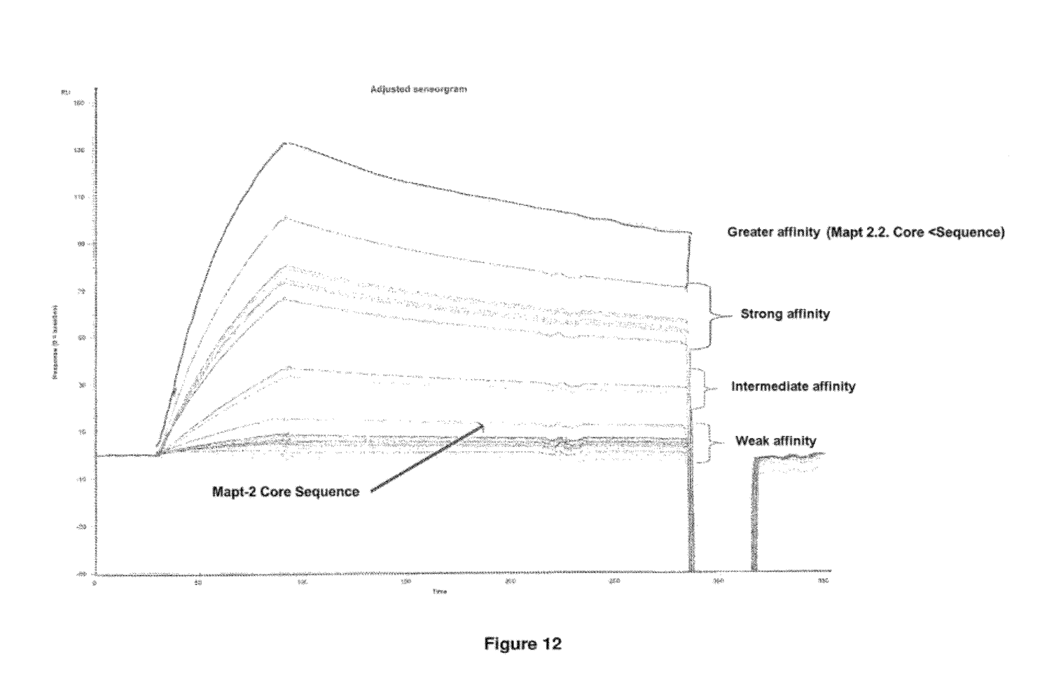

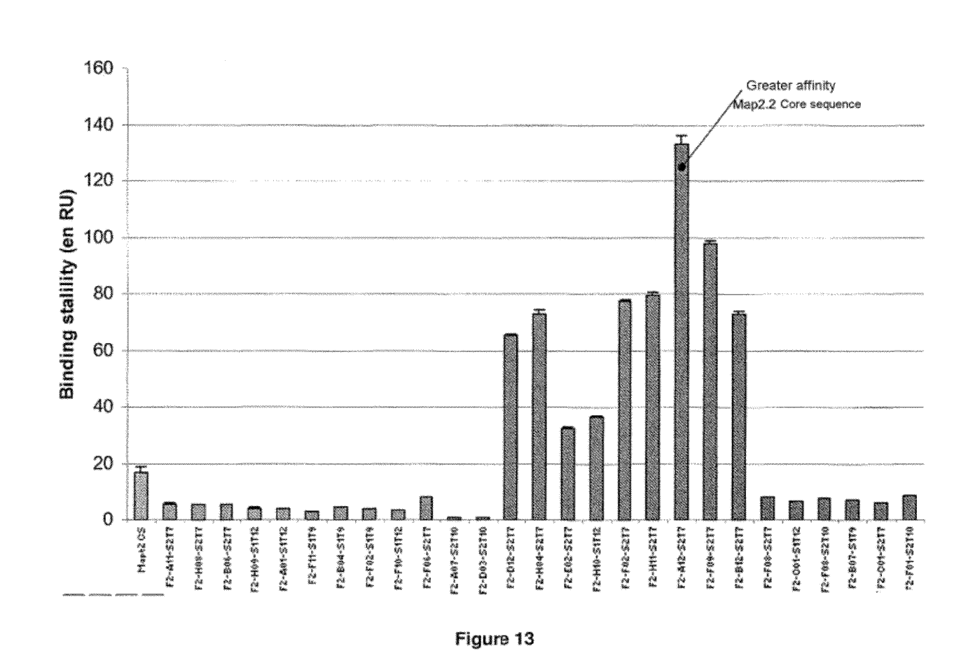

The results are presented in FIGS. 12 and 13.

FIG. 12 illustrates the curves of binding of a series of 27 nucleic aptamers of the invention to human plasma factor VII immobilized on a substrate, in a test according to the surface plasmon resonance technique. On the x-axis: time, expressed in seconds; along the y-axis: resonance signal, expressed in arbitrary resonance units.

FIG. 13 illustrates the individual values of the signal for stable binding of each of the 27 aptamers tested. Along the x-axis: each of the 27 aptamers tested; along the y-axis: resonance signal, expressed in arbitrary resonance units.

Four groups of aptamers which differ by virtue of their affinity for human factor VII are observed.

Mapt2 “core sequence” is found in the low affinity group. One representative of the family 2 has a much higher affinity than the others. This aptamer is called Mapt2.2 and has the following “core sequence”:

(SEQ ID No. 33)
5′CCGCACGCTACGCGCATGAACCCGCGCACACGACTTGAAGTAGC3′.

The results obtained show, nevertheless, that all the nucleic aptamers tested bind with significant affinity to human plasma factor VII.

### Example 12

### Method for Purifying Human Plasma Factor VII

### A. Materials and Methods

### A.1. Affinity Chromatography Substrate

Affinity gel material on which was immobilized the “Mapt-2 core” aptamer coupled directly to biotin, without a spacer chain between the aptamer and the biotin. The aptamer is immobilized on a streptavidin gel (supplier Novagen) by means of a 5′-terminal biotin, with a theoretical ligand density of 0.4 mg/ml: volume 1 ml packed in an XK16 column (GE).

The aptamer used is the aptamer of sequence SEQ ID No. 20. This starting product comprises impurities, truncated forms of factor VII and degraded forms of factor VII. A degraded form of factor VII may comprise a form of factor VII in which the gamma-carboxylation is modified.

### A.3. Purification Protocol

Gel equilibration: 0.050 M Tris-HCl, 0.010 M CaCl₂, 0.05 mM MgCl₂, pH 7.5,
elution: 0.020 M Tris-HCl, 0.010 M EDTA, pH 7.5,
240 μg of human plasma FVII purified to 98% is injected with a flow rate of 0.5 ml/min in equilibration buffer.

After detection of the nonretained peak, 2 column volumes of elution buffer are injected.

The protein peaks are detected by measuring the absorbance value at the wavelength of 280 nanometers.

### B. Results

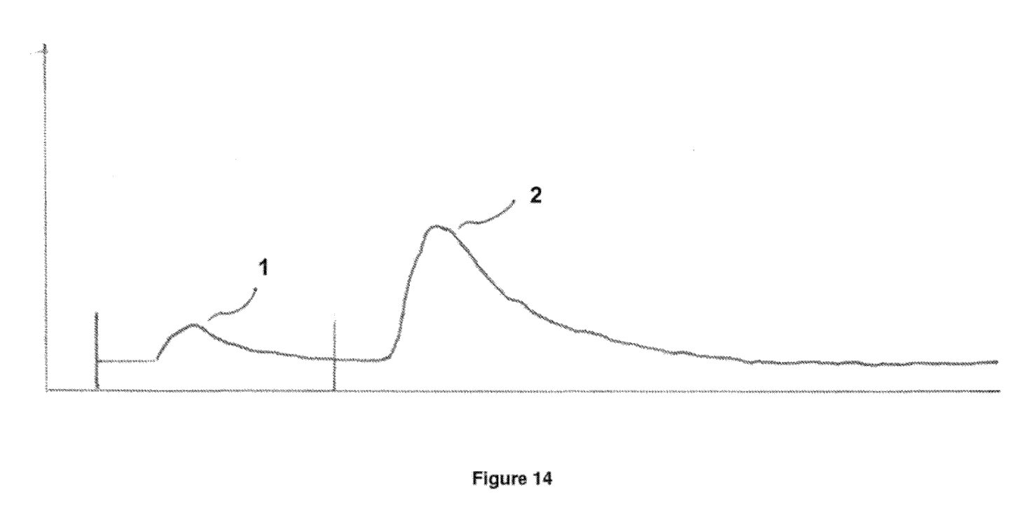

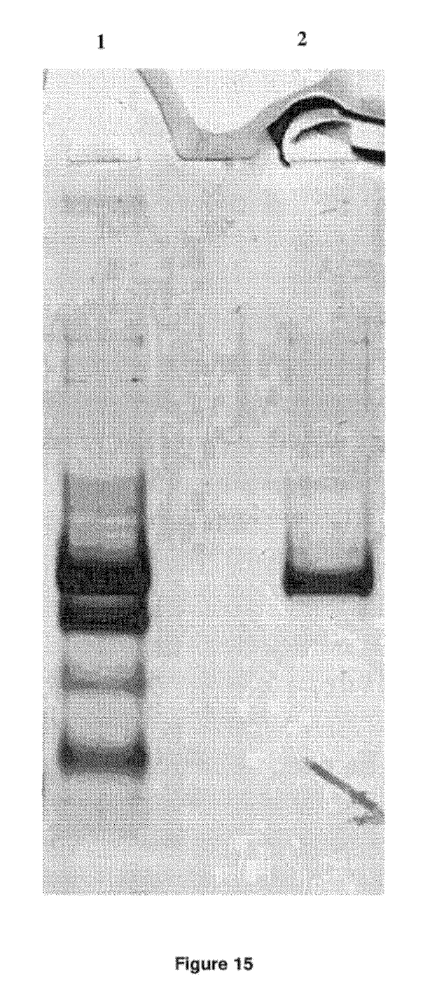

The results are illustrated in FIGS. 14 and 15.

FIG. 14 represents the curve of the values of the measurement of absorbance at 280 nm as a function of time. In FIG. 14, peak No. 1 corresponds to the fraction of the starting product which was not retained on the column. Peak No. 2 corresponds to the elution fraction.

The starting product and also the eluted product were analyzed by SDS PAGE with silver nitrate staining in order to visualize the elimination of the impurities. FIG. 15 represents this gel: lane No. 1 corresponds to the fraction of the starting product and lane No. 2 to the elution fraction. Despite the considerable purity of the starting product, it is noted that the eluted fraction no longer contains contaminants or degraded forms.

The results of FIGS. 14 and 15 show that the aptamer of sequence SEQ ID No. 20 is capable of binding human FVII and of specifically eluting it in the presence of EDTA.

### Example 13

### Absence of Binding of the Aptamer to Rabbit FVII

### A. Materials and Methods

### A.1. Affinity Chromatography Substrate

Affinity gel coupled to streptavidin, on which the aptamer of sequence SEQ ID No. 86 was immobilized by means of a spacer chain (supplier Novagen) via a 5′-terminal biotin, with a theoretical ligand density of 0.35 mg/ml: volume 1 ml.

The aptamer used is the aptamer of sequence SEQ ID No. 86.

### A.2. Starting Product

Eluate of hydroxyapatite enriched in rabbit FVII obtained by purification from rabbit plasma, contact time 10 minutes, flow rate 0.5 ml/min.

### A.3. Purification Protocol

Gel equilibration: 0.050 M Tris-HCl, 0.010 M CaCl₂, 0.05 mM MgSO₃, pH 7.5,
elution: 0.050 M Tris-HCl, 0.010 M EDTA, pH 7.5,
36 μg are injected into the gel with a contact time of 10 minutes. The elution is carried out by injecting 2 ml of elution buffer.

The protein peaks are detected by measuring the value of absorbance at the wavelength of 280 nanometers.

### A.4. Protocols for Analysis of the Fractions in Terms of Proteins and in Terms of Factor VII

The fractions are analyzed for their amidolytic activity by chromogenic assay using a Stago kit according to the supplier's recommendations (factor VIIa StatClot kit). The amidolytic activity is then converted to pg of FVII contained in said fraction.

### B. Results

The results are illustrated in table 4 below.

TABLE 4

| | | | | Amount | | Step |
|---|---|---|---|---|---|---|
| | Proteins | FVIIam | Volume | FVII | Purity | yield |
| Steps | (mg/ml) | (IU/ml) | (ml) | (μg) | (%) | (%) |
| Starting material | 1.38 | 57 | 2.5 | 71 | 2% | 100% |
| Dialyzed eluate | 0.97 | 21.8 | 3.4 | 36.7 | 1% | 52% |
| Mapt-2 eluate | NA | 0.04 | 4.0 | 0.08 | NA | 0.2% |

The results in table 4 show that the rabbit factor VII is not retained on the affinity gel on which the Mapt-2 aptamer of sequence SEQ ID No. 86 is immobilized.

### Example 14

### Specific Embodiments of a Protocol for Interaction of Human Factor VII with an Aptamer on Biacore (Resistance to NaCl)

### A. Materials and Methods

A solid substrate on which molecules of the nucleic aptamer of the invention of sequence SEQ ID No. 86, also denoted herein “Mapt2”, were immobilized, was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (Series S sensor Chip SA, GE)

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 3772 RU (1 RU corresponds approximately to 1 pg of product immobilized per mm²).

Purified transgenic human FVII obtained from transgenic rabbit milk (FVII HPTG, purity: 98%) was diluted in running buffer (50 mM Tris, 50 mM NaCl, 10 mM CaCl₂, 4 mM MgCl₂, pH 7.4) so as to obtain a sample having an FVII concentration of 200 mM.

The sample was injected onto the chip (solid substrate) containing the Mapt2 aptamer immobilized by means of a biotin-streptavidin interaction. Next, buffers containing increasing concentrations of NaCl were injected onto the solid substrate (3 series of injections ranging from 1M NaCl to 3M NaCl). All the injections were carried out with a flow rate of 30 μl/min for 60 sec after the injection. After the 3 series of injections with the 3 buffers containing NaCl, elution buffer (10 mM EDTA) was then injected for 75 sec with a flow rate of 30 μl/min in order to detach the FVII HPTG from the aptamer.

These analyses are carried out with the RPS Biacore T100 apparatus (GE). The modeling of the interactions recorded is carried out using the Biaevaluation software (GE).

The curves of binding of the immobilized Mapt2 aptamer to the transgenic human FVII were calculated with the dedicated module of the Biacore® control software, version 1.2.

The results of the binding of the Mapt2 aptamer to human FVII made it possible to determine that the binding of the Mapt2 aptamer to human FVII is not detrimentally modified by the injection of the buffers containing NaCl.

### B. Results

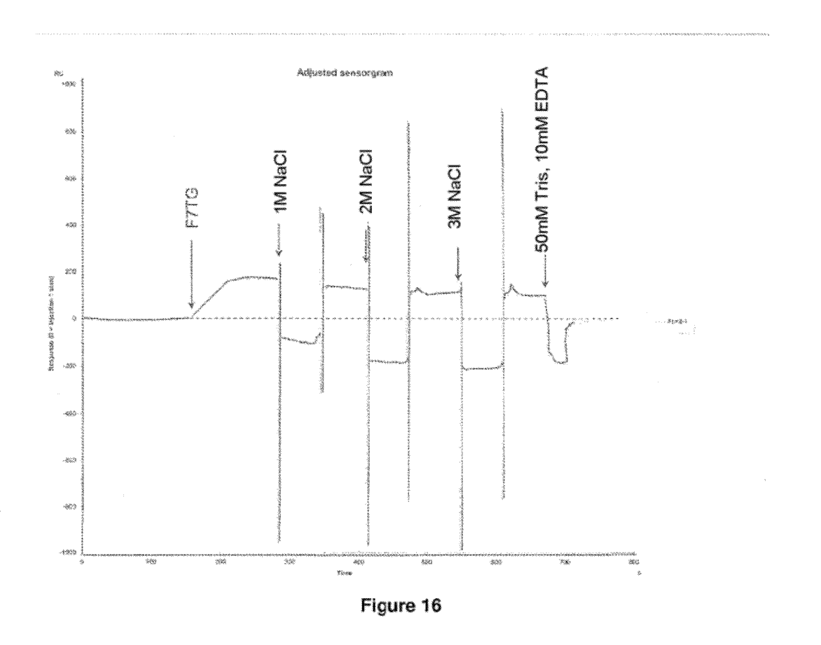

The results are illustrated in FIG. 16.

### Example 15

### Specific Embodiments of a Protocol for Interaction of Human Factor VII with an Aptamer on Biacore (Resistance to Propylene Glycol)

### A. Materials and Methods

A solid substrate on which molecules of the nucleic aptamer of the invention of sequence SEQ ID No. 86, also denoted herein “Mapt2”, were immobilized, was produced. Prior to its binding to the solid substrate, the 5′ end of the Mapt2 aptamer was chemically coupled to a spacer chain consisting of 4 molecules of PEG(C18). Then, the free end of the spacer chain, opposite the end coupled to the aptamer, was coupled to a biotin molecule.

A solid substrate containing immobilized streptavidin molecules is provided (Series S sensor Chip SA, GE).

The solid substrate above was then brought into contact with the aptamer compounds above in order to immobilize the nucleic acids of sequence SEQ ID No. 86, by noncovalent association between the streptavidin molecules of the substrate and the biotin molecules of the aptamer compounds.

The Mapt2 aptamer is thus immobilized with a degree of immobilization of 5319 RU (1 RU corresponds approximately to 1 pg of product immobilized per mm²).

Purified transgenic human FVII obtained from transgenic rabbit milk (FVII HPTG, purity: 98%) was diluted in running buffer (50 mM Tris, 10 mM CaCl₂, 4 mM MgCl₂, pH 7.4) so as to obtain a sample having an FVII concentration of 200 mM.

The sample was injected onto the chip (solid substrate) containing the Mapt2 aptamer immobilized by means of a biotin-streptavidin interaction. Next, a buffer containing 50% of propylene glycol was injected onto the solid substrate. All the injections were carried out with a flow rate of 30 μl/min for 60 sec after the injection. After the injection with the buffer containing 50% propylene glycol, elution buffer (10 mM EDTA) was then injected for 75 sec with a flow rate of 30 μl/min in order to detach the FVII HPTG from the aptamer.

These analyses are carried out with the RPS Biacore T100 apparatus (GE). The modeling of the interactions recorded is carried out using the Biaevaluation software (GE).

The curves of binding of the immobilized Mapt2 aptamer to the transgenic human FVII were calculated with the dedicated module of the Biacore® control software, version 1.2.

The results of binding of the Mapt2 aptamer to human FVII made it possible to determine that the binding of the Mapt2 aptamer to human FVII is not detrimentally modified by the injection of the buffer containing propylene glycol.

### B. Results

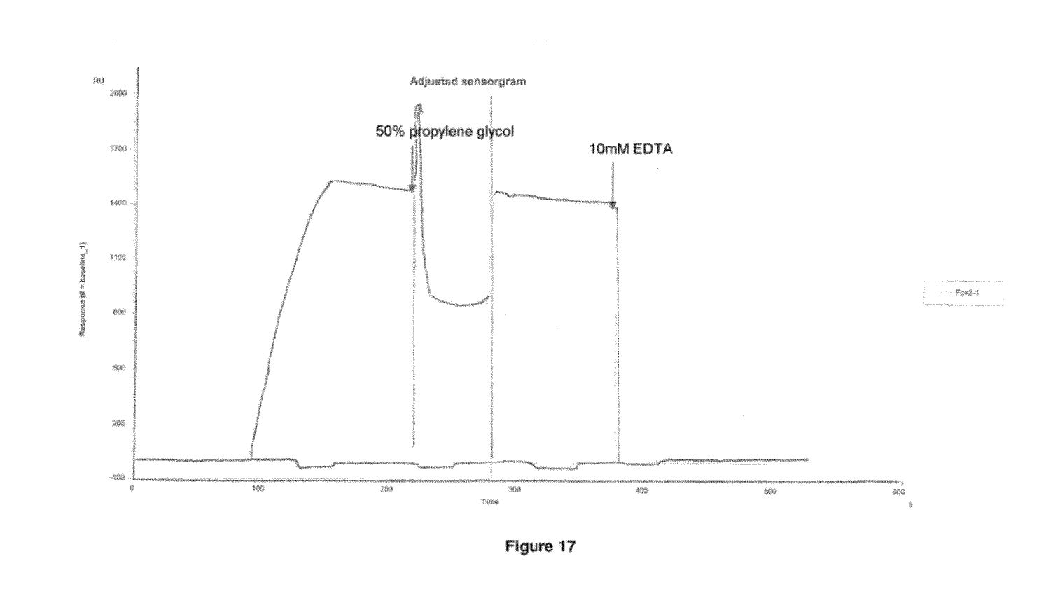

The results are illustrated in FIG. 17.

1-20. (canceled)

21. A nucleic acid which binds specifically to human factor VII/VIIa, said nucleic acid comprising at least 15 consecutive nucleotides of a polynucleotide having at least 40% nucleotide identity with the nucleic acid of formula (I) below:
5′-[SEQ ID No. 1]x-[SEQ ID No. X]-[SEQ ID No. 2]y-3′ (I),
in which:
“SEQ ID No. X” is chosen from the group consisting of the nucleic acids having at least 40% nucleotide identity with at least one of the sequences SEQ ID No. 3 to SEQ ID No. 85 and SEQ ID No. 87 to SEQ ID No. 100,
“x” is an integer equal to 0 or 1, and
“y” is an integer equal to 0 or 1.

22. The nucleic acid as claimed in claim 21, wherein the sequence SEQ ID No. X is chosen from the group consisting of the nucleic acids having at least 80% nucleotide identity, with at least one of the sequences SEQ ID No. 3 to SEQ ID No. 85 and SEQ ID No. 87 to SEQ ID No. 100.

23. The nucleic acid as claimed in claim 21, wherein the sequence SEQ ID No. X is chosen from the group consisting of the sequences SEQ ID No. 3 to SEQ ID No. 85 and SEQ ID No. 87 to SEQ ID No. 100.

24. The nucleic acid as claimed in claim 21, further defined as comprising at least 15 consecutive nucleotides of a polynucleotide having at least 40% nucleotide identity with a nucleic acid chosen from the group consisting of the nucleic acids of sequences SEQ ID No. 3 to SEQ ID No. 85 and SEQ ID No. 87 to SEQ ID No. 100.

25. The nucleic acid as claimed in claim 21, further defined as having a capacity to bind specifically to human factor VII/VIIa expressed by the following condition (A):
human Kd/nonhuman Kd<0.01 (A),
in which:
“human Kd” is the dissociation constant of a nucleic acid of formula (I) for human factor VII/VIIa, estimated in molar units, and
“nonhuman Kd” is the dissociation constant of said nucleic acid of formula (I) for a nonhuman factor VII/VIIa, expressed in the same molar units.

26. The nucleic acid as claimed in claim 21, further defined as having a dissociation constant value for human factor VII/VIIa of at most 500 nM.

27. The nucleic acid as claimed in claim 21, wherein the binding thereof to human factor VII/VIIa can be dissociated by bringing into contact with a metal-cation-chelating agent.

28. A compound which binds specifically to human factor VII/VIIa, further defined as it is of formula (II) below:
[SPAC]-[NUCL] (II), in which:
[SPAC] signifies a spacer chain, and
[NUCL] signifies a nucleic acid which binds specifically to human factor VII/VIIa comprising at least 15 consecutive nucleotides of a polynucleotide having at least 40% nucleotide identity with the nucleic acid of formula (I) as defined in claim 21.

29. A compound which binds specifically to human factor VII/VIIa, further defined as it is of formula (III) below:
[FIX]-[SPAC]-[NUCL] (III), in which:
[FIX] signifies a compound for immobilization on a substrate,
[SPAC] signifies a spacer chain, and
[NUCL] signifies a nucleic acid which binds specifically to human factor VII/VIIa comprising at least 15 consecutive nucleotides of a polynucleotide having at least 40% nucleotide identity with the nucleic acid of formula (I) as defined in claim 21.

30. A complex between (i) a nucleic acid as claimed in claim 21, and (ii) a human factor VII/VIIa.

31. A substrate for the immobilization of human factor VII/VIIa, further defined as comprising a solid substrate material onto which a plurality of nucleic acids as claimed in claim 21 is grafted.

32. A method for immobilizing human factor VII/VIIa on a substrate, comprising a step during which a sample comprising human factor VII/VIIa is brought into contact with a substrate as claimed in claim 31.

33. A method for purifying human factor VII/VIIa comprising the following steps:
a) bringing a sample comprising human factor VII/VIIa into contact with a nucleic acid as claimed in claim 21, in order to form a complex between (i) said nucleic acid, and (ii) the human factor VII/VIIa, and
b) releasing the human factor VII/VIIa from the complex formed in step a) and recovering the purified human factor VII/VIIa.

34. A method for detecting the presence of human factor VII/VIIa in a sample, comprising the following steps:
a) bringing a nucleic acid as claimed in claim 21 into contact with said sample; and
b) detecting the formation of complexes between (i) said nucleic acid or said compound or said substrate and (ii) the factor VII/VIIa.

35. A pharmaceutical composition comprising a nucleic acid as claimed in any claim 21, in combination with one or more pharmaceutically acceptable excipients.

36. A complex between (i) a compound as claimed in claim 28, and (ii) a human factor VII/VIIa.

37. A substrate for the immobilization of human factor VII/VIIa, further defined as comprising a solid substrate material onto which a compound as claimed in claim 28 is grafted.

38. A method for purifying human factor VII/VIIa comprising the following steps:
a) bringing a sample comprising human factor VII/VIIa into contact with a compound as claimed in claim 28, in order to form a complex between (i) said compound and (ii) the human factor VII/VIIa, and
b) releasing the human factor VII/VIIa from the complex formed in step a) and recovering the purified human factor VII/VIIa.

39. A method for purifying human factor VII/VIIa comprising the following steps:
a) bringing a sample comprising human factor VII/VIIa into contact with a substrate as claimed in claim 31, in order to form a complex between (i) said substrate and (ii) the human factor VII/VIIa, and
b) releasing the human factor VII/VIIa from the complex formed in step a) and recovering the purified human factor VII/VIIa.

40. The nucleic acid as claimed in claim 22, wherein the sequence SEQ ID No. X is chosen from the group consisting of the sequences SEQ ID No. 3 to SEQ ID No. 85 and SEQ ID No. 87 to SEQ ID No. 100.
